(19) United States
(12) Patent Application Publication
(10) Pub. No.: US 2012/0316176 A1
(43) Pub. Date: Dec. 13, 2012

(54) DERIVATIVES OF 7,8-DIHYDRO-1H-IMIDAZO[2,1-b] PURIN-4(5H)-ONE and METHODS OF USE THEREOF

(76) Inventors: Deen Tulshian, Lebanon, NJ (US); Michael F. Czarniecki, Watchung, NJ (US); Julius Matasi, Monmouth Junction, NJ (US); Stephanie N. Brumfield, Rahway, NJ (US)

(21) Appl. No.: 13/517,442

(22) PCT Filed: Dec. 16, 2010
(86) PCT No.: PCT/US10/60632

§ 371 Date: Jun. 20, 2012

(30) Foreign Application Priority Data
Dec. 22, 2009 (US) 61289068

(51) Int. Cl. C07D 487/14; A61P 11/00; A61P 19/02; A61P 11/06; A61K 31/519; A61P 29/00

(52) U.S. Cl. 514/252.02; 544/251; 514/267; 514/255.05; 544/238

(57) ABSTRACT

The present invention relates to derivatives of 7,8-dihydro-1H-imidazo[2,1-b]purin-4(5H)-one, compositions thereof and methods of use thereof for treating or preventing pain or an inflammatory disease.

### FIELD OF THE INVENTION

The present invention relates to derivatives of 7,8-dihydro-1H-imidazo[2,1-b]purin-4(5H)-one, compositions thereof and methods of use thereof for treating or preventing pain or an inflammatory disease.

### BACKGROUND OF THE INVENTION

P2X₇ is a ligand-gated ion channel that responds to elevated ATP levels, commonly found at sites of injury and inflammation, by mediating the cellular efflux of K⁺ and the influx of Ca⁺⁺ and Na⁺. P2X₇ is expressed by leukocytes, in particular by T cells, B cells, neutrophils and monocytes/macrophages, and by chondrocytes, synoviocytes, microglia and astrocytes. The major downstream effector function of P2X₇ activation is the processing and release of mature forms of the proinflammatory cytokines IL-1β and 1L-18 which involves the activation of caspase-1 (ICE). This occurs through the action of NALP3/cryopyrin-dependent inflammasomes. Activation of P2X₇ also has been shown to increase levels of other mediators of inflammation including MMPs, PGE₂ and TNF-α, although the mechanisms for these effects are not as well studied. There are data supporting a role for P2X₇ in signaling cascades such as NF-κB and these pathways might provide a link to non-inflammasome-based mediator production. Current knowledge of P2X₇ inhibitors indicates that antagonism of P2X₇ in vivo reduces inflammatory cytokines and inflammation and reduces both inflammatory hyperalgesia and neuropathic pain.

Rheumatoid arthritis is another disease linked to the activity of P2X₇. Rheumatoid arthritis is characterized by significant synovial inflammation and destruction of extracellular matrix and articular structures including cartilage and bone. Cytokine pathways, including TNF-α, IL-1β, IL-18 and IL-6, are thought to play significant roles in this process. This has been clinically validated for TNF-α and IL-6. Inflamed synovium contains a variety of cells, including macrophage, T cells, B cells, synoviocytes, fibroblasts and chondrocytes which are known to express P2X₇ and contribute to the production of these cytokines. Therefore, P2X₇ antagonists are potentially useful as they may inhibit the inflammatory cascade observed in rheumatoid arthritis.

The expression of P2X₇ on immune cells and its role in cytokine production also suggest the potential utility of P2X₇ antagonists in the treatment of chronic obstructive pulmonary disease (COPD), asthma and inflammatory bowel disease (IBD). P2X₇-expressing cells including macrophage, T cells and neutrophils through their mediators such as IL-18 and proteases play important roles in the cascade of events leading to lung tissue destruction and reduced lung function in COPD patients. Similarly, macrophage and T cells and their mediators play important roles in the path physiology of asthma and IBD.

There remains a need in the art for novel compounds which are useful for treating inflammatory diseases and pain. This invention addresses that need.

### SUMMARY OF THE INVENTION

In one aspect, the present invention provides compounds having the formula:

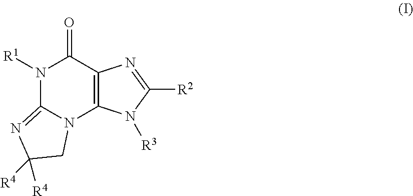

and pharmaceutically acceptable salts, solvates, esters and prodrugs thereof, wherein:

R¹ is —C₁-C₆ alkyl, alkenyl, or -alkylene-O—C₁-C₆ alkyl;

R² is —C₁-C₆alkyl, —C₂-C₈alkynyl, —C₆-C₁₀aryl, -alkylene-C₆-C₁₀aryl, —C₃-C₁₀heterocycloalkyl or —C₅-C₁₀heteroaryl, any of which may be optionally substituted with R⁵;

R³ is —C₁-C₆alkyl, -alkylene-C₆-C₁₀aryl, C₃-C₁₀cycloalkyl, -alkylene-C₃-C₁₀cycloalkyl or -alkylene-C₃-C₁₀heterocycloalkyl, wherein an aryl, cycloalkyl or heterocycloalkyl group can be optionally substituted with R⁷;

each occurrence of R⁴ is independently H, —C₁-C₆ alkyl, C₆₋₁₀aryl, or C₃-C₁₀cycloalkyl, or both R⁴ groups together with the carbon atom to which they are attached, join to form a 3- to 7-membered cycloalkyl group, which can be optionally fused with a benzene ring;

R⁵ represents from 1 to 3 groups, each independently selected from —C₂-C₆alkenyl, —C₂-C₆alkynyl, —C₃-C₁₀cycloalkyl, —C₆-C₁₀aryl, —C₃-C₁₀heterocycloalkyl, —C₆-C₁₀heteroaryl, halo, —CN, —C(O)OR⁶, —C(O)R⁶, —C(O)N(R⁶)₂, —S(O)₂NHR⁶, —OH, —O-alkyl, haloalkyl, O—(CH₂)ₙhaloalkyl, and —NHC(O)N(R⁶)₂, where an alkynyl, aryl, heterocycloalkyl or heteroaryl group may be optionally substituted with up to 3 groups, each independently selected from —C₁-C₆ alkyl, —C₃-C₁₀cycloalkyl, halo, haloalkyl, CN, OH, —O-alkyl, —C(O)OR⁶, —C(O)R⁶, NHC(O)C₁-C₆ alkyl, and —(CH₂)ₙC(O)N(R⁶)₂;

each occurrence of R⁶ is independently H, —C₁-C₆ alkyl, —C₆-C₁₀aryl or —C₃-C₁₀heterocycloalkyl, wherein an aryl or heterocycloalkyl group can be optionally substituted with up to 3 groups, each independently selected from —C₁-C₆alkyl, —OC₁-C₆alkyl, halo, —CN, haloalkyl, -alkylene-C(O)N(R⁸)₂, —C(O)N(R⁸)₂, —C(O)OR⁸, —C(O)C₃-C₁₀heterocycloalkyl, —C(O)C₁-C₆alkyl or —N(R⁸)₂;

R⁷ represents from 1 to 3 groups, each independently selected from —C₁-C₆ alkyl, halo, —C₆-C₁₀aryl, NO₂, —OC₁-C₆alkyl, —N(R⁸)₂, —C(O)OR⁸, —C(O)N(R⁸)₂ and haloalkyl;

each occurrence of R⁸ is independently H, —C₁-C₆ alkyl or —C₆-C₁₀aryl and

n is 0-1.

The Compounds of Formula (I) and pharmaceutically acceptable salts, solvates, esters and prodrugs thereof, can be useful for treating or preventing pain or an inflammatory disease (each being a “Condition”) in a patient.

The invention also provides pharmaceutical compositions comprising an effective amount of one or more Compounds of Formula (I) or a pharmaceutically acceptable salt, solvate, ester or prodrug thereof, and a pharmaceutically acceptable carrier. The compositions can be useful for treating or preventing a Condition in a patient.

The invention further provides pharmaceutical compositions comprising an effective amount of one or more Compounds of Formula (I) or a pharmaceutically acceptable salt, solvate, ester or prodrug thereof, and a pharmaceutically acceptable carrier. The compositions can be useful for treating or preventing a Condition in a patient.

The invention also provides methods for treating or preventing a Condition in a patient, comprising administering to the patient an effective amount of one or more Compounds of Formula (I).

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

In an embodiment, the present invention provides Compounds of Formula (I), pharmaceutical compositions comprising one or more Compounds of Formula (I), and methods of using the Compounds of Formula (I) for treating or preventing a Condition in a patient.

### DEFINITIONS AND ABBREVIATIONS

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “effective amount” as used herein, refers to an amount of a Compound of Formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkyl, aryl, cycloalkyl, haloalkyl, —CN, —OH, —O-alkyl, —O-aryl, -alkylene-O-alkyl, —S-alkyl, —NH(cycloalkyl), —O—C(O)-alkyl, —O—C(O)-aryl, —O—C(O)-cycloalkyl, —C(O)OH, —C(O)O-alkyl, —C(O)N(R⁵⁰)₂ or —N(R⁵⁰)₂, wherein each occurrence of R⁵⁰ is independently H, alkyl or aryl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkyl, aryl, cycloalkyl, haloalkyl, —CN, —OH, —O-alkyl, —O-aryl, -alkylene-O-alkyl, —S-alkyl, —NH(cycloalkyl), —O—C(O)-alkyl, —O—C(O)-aryl, —O—C(O)-cycloalkyl, —C(O)OH, —C(O)O-alkyl, C(O)N(R⁵⁰)₂ and —N(R⁵)₂, wherein each occurrence of R⁵⁰ is independently H, alkyl or aryl. In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkyl, aryl, cycloalkyl, haloalkyl, —CN, —OH, —O-alkyl, —O-aryl, -alkylene-O-alkyl, —S-alkyl, —NH(cycloalkyl), —O—C(O)-alkyl, —O—C(O)-aryl, —O—C(O)-cycloalkyl, —C(O)OH, —C(O)O-alkyl, —C(O)N(R⁵⁰)₂ and —N(R⁵⁰)₂, wherein each occurrence of R⁵⁰ is independently H, alkyl or aryl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. An alkylene group may be unsubstituted or substituted by one or more substituents which may be the same or different, each substituent being independently selected from the group consisting of halo, alkyl, aryl, cycloalkyl, haloalkyl, —CN, —OH, —O-alkyl, —O-aryl, -alkylene-O-alkyl, —S-alkyl, —NH(cycloalkyl), —O—C(O)-alkyl, —O—C(O)-aryl, —O—C(O)-cycloalkyl, —C(O)OH, —C(O)O-alkyl, —C(O)N(R⁵⁰)₂ and —N(R⁵⁰)₂, wherein each occurrence of R⁵⁰ is independently H, alkyl or aryl. In one embodiment, an alkylene group is unsubstituted. In another embodiment, an alkylene group has from 1 to about 6 carbon atoms. In another embodiment, an alkylene group is branched. In still another embodiment, an alkylene group is linear.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH₂CH═CH—, —CH₂CH═CHCH₂—, —CH═CHCH₂CH₂—, —CH₂CHCH═CH—, —CH(CH₃)CH═CH— and —CH═C(CH₃)CH₂—. In one embodiment, an alkenylene group has from 2 to about 6 carbon atoms.

The term “alkynylene,” as used herein, refers to an alkynyl group, as defined above, wherein one of the alkynyl group's hydrogen atoms has been replaced with a bond. Non-limiting examples of alkynylene groups include —C≡C—, —CH₂C≡C—, —CH₂C≡CCH₂—, —C≡CCH₂CH₂—, —CH₂CHC≡C—, —CH(CH₃)C≡C— and —C≡CCH₂—. In one embodiment, an alkynylene group has from 2 to about 6 carbon atoms

“Aryl” means an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl. In another embodiment, an aryl group is naphthyl. In another embodiment, an aryl group is a phenyl group which is substituted with one F atom. In still another embodiment, an aryl group is a phenyl group which is substituted with two F atoms.

The term “arylene,” as used herein, refers to an aryl group, as defined above, wherein one of the aryl group's hydrogen atoms has been replaced with a bond. Non-limiting examples of arylene groups include:

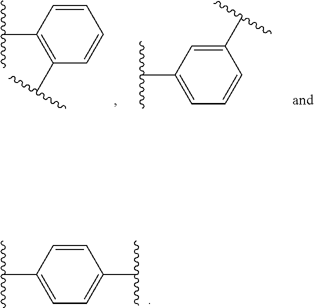

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 5 to about 7 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “cycloalkylene,” as used herein, refers to a cycloalkyl group, as defined above, wherein one of the cycloalkyl group's hydrogen atoms has been replaced with a bond. Non-limiting examples of cycloalkylene groups include:

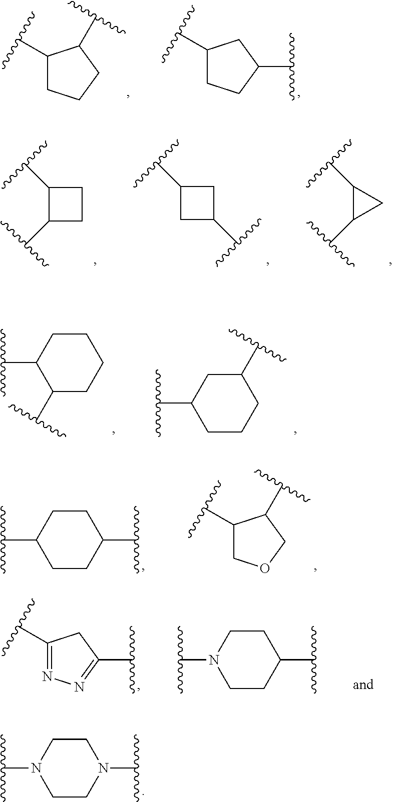

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which has been fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl group is unsubstituted. In another embodiment, a heteroaryl group is a 5-membered heteroaryl. In another embodiment, a heteroaryl group is a 6-membered heteroaryl.

The term “heteroarylene,” as used herein, refers to a heteroaryl group, as defined above, wherein one of the heteroaryl group's hydrogen atoms has been replaced with a bond. Non-limiting examples of heteroarylene groups include:

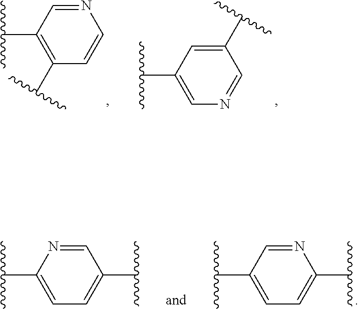

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

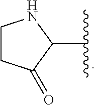

In one embodiment, a heterocycloalkyl group is unsubstituted. In another embodiment, a heterocycloalkyl group is a 5-membered heterocycloalkyl. In another embodiment, a heterocycloalkyl group is a 6-membered heterocycloalkyl.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -alkylene-heteroaryl, -arylene-alkyl, alkenylene-heteroaryl, alkynylene-heteroaryl, hydroxy, hydroxyalkyl, haloalkyl, —O-alkyl, -alkylene-O-alkyl, —O-aryl, aralkoxy, acyl, aroyl, halo, nitro, cyano, SF₅, carboxy, —C(O)O-alkyl, —C(O)O-aryl, —C(O)O-alkelene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂S(O)₂— and —SO₂NY₁Y₂, wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

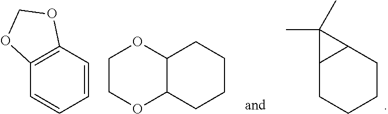

“Halo” means —F, —Cl, —Br or —I. In one embodiment, halo is —Cl or —F. In another embodiment, halo is —F.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g., from a reaction mixture), or natural source or combination thereof. Thus, the term “purified”, “in purified form” or “in isolated and purified form” for a compound refers to the physical state of the compound after being obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Compound of Formula (I) or a pharmaceutically acceptable salt, hydrate or solvate of the compound. The transformation may occur by various mechanisms (e.g., by metabolic or chemical processes), such as, for example, through hydrolysis in blood.

For example, if a Compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-(C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Compound of Formula (I) incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or —O—C₁₋₄alkyl or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Compounds of Formula (I) can form salts which are also within the scope of this invention. Reference to a Compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Compounds of Formula (I) may be atropisomers (e.g., substituted biaryls) and are considered as part of this invention. Enantiomers can also be directly separated using chiral chromatographic techniques.

It is also possible that the Compounds of Formula (I) may exist in different tautomeric forms, and all such forms are embraced within the scope of the invention. For example, all keto-enol and imine-enamine forms of the compounds are included in the invention. It should also be noted that tautomeric forms such as, for example, the moieties:

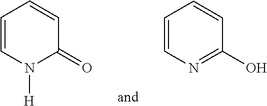

are considered equivalent in certain embodiments of this invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). If a Compound of Formula (I) incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt” “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Compounds of Formula (I) (e.g., those labeled with ³H and ¹⁴C) are useful in compound and/or substrate tissue distribution assays. In one embodiment, tritiated (i.e., ³H) and carbon-14 (i.e., ¹⁴C) isotopes are employed for their ease of preparation and detectability. In another embodiment, substitution with heavier isotopes such as deuterium (i.e., ²H) may afford certain therapeutic advantages resulting from greater metabolic stability (e.g., increased in vivo half-life or reduced dosage requirements). In one embodiment, a Compound of Formula (I) has one or more of its hydrogen atoms replaced with a deuterium atom.

Isotopically labelled compounds of Formula (I) can generally be prepared by following procedures analogous to those disclosed in the Schemes and/or in the Examples herein below, by substituting an appropriate isotopically labelled reagent for a non-isotopically labelled reagent.

Polymorphic forms of the Compounds of Formula (I), and of the salts, solvates, hydrates, esters and prodrugs of the Compounds of Formula (I), are intended to be included in the present invention.

Polymorphic forms of the Compounds of Formula (I), and of the salts, solvates, hydrates, esters and prodrugs of the Compounds of Formula (I), are intended to be included in the present invention.

The following abbreviations are used below and have the following meanings: t-butyl is tertiary butyl, DIPEA is diisopropylethylamine, DMA is N,N-dimethylacetamide, DME is dimethoxyethane, DMF is N,N -dimethylformamide, DMSO is dimethylsulfoxide, EtOAc is ethyl acetate, EtOH is ethanol, Et₃N is triethylamine, i-Pr is isopropyl, LCMS is liquid chromatography mass spectrometry, MeOH is methanol, NaOMe is sodium methoxide, NBS is N-bromosuccinimide, NMP is N-methylpyrrolidone, NMR is nuclear magnetic resonance, Ph is phenyl and THF is tetrahydrofuran.

### The Compounds of Formula (I)

In one embodiment, the present invention provides compound having the formula:

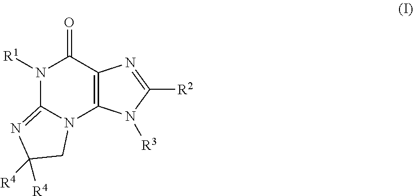

and pharmaceutically acceptable salts, solvates, esters and prodrugs thereof, wherein R¹, R², R³ and R⁴ are defined above for the Compounds of Formula (I).

In one embodiment, for the Compounds of Formula (I), variables R¹, R², R³ and R⁴ are selected independently of each other.

In another embodiment, a Compound of Formula (I) is in purified form.

In another embodiment, a Compound of Formula (I) can be an antagonist of P2X₇.

In one embodiment, the Compounds of Formula (I) have the formula (Ia):

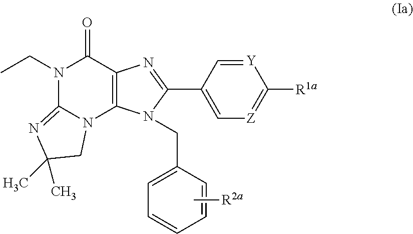

wherein:

Y is N or —C(R^{3a})—;

Z is N or —C(R^{3a})—;

R^{1a} is —O-alkyl, —O-haloalkyl, cyclohexyl, piperidinyl, pyridyl, pyridazinyl, pyrazinyl, phenyl, piperidinyl or oxadiazolyl, wherein a piperidinyl, pyridyl, pyridazinyl, pyrazinyl, phenyl, piperidinyl or oxadiazolyl group can be unsubstituted or optionally substituted with up to 2 groups, which can be the same or different, and are selected from alkyl, —O-alkyl, —CN, halo or —C(O)O-alkyl;

R^{2a} represents at least one —F group and up to a maximum of three —F groups; and

each occurrence of R^{3a} is independently H, F, OCHF₂, or —CN.

In one embodiment, for the Compounds of Formula (Ia), Y is N and Z is —C(R^{3a})—.

In another embodiment, for the Compounds of Formula (Ia), Z is N and Y is —C(R^{3a})—.

In another embodiment, for the Compounds of Formula (Ia), Y and Z are each N.

In still another embodiment, for the Compounds of Formula (Ia), the ring containing Y and Z has the structure:

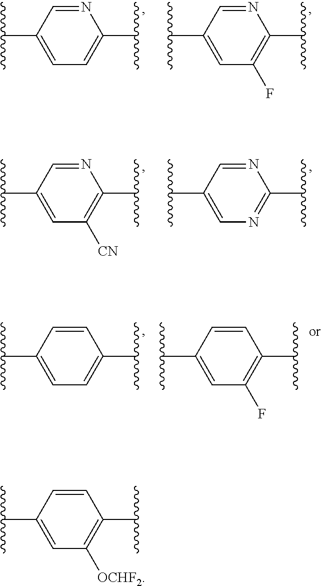

In one embodiment, for the Compounds of Formula (Ia), R^{1a} is —O-alkyl.

In another embodiment, for the Compounds of Formula (Ia), R^{1a} is methoxy.

In another embodiment, for the Compounds of Formula (Ia), R^{1a} is methoxy or —OCHF₂.

In still another embodiment, for the Compounds of Formula (Ia), R^{1a} is piperidinyl, which can be unsubstituted or optionally substituted with a —C(O)O-alkyl group.

In another embodiment, for the Compounds of Formula (Ia), R^{1a} is pyridyl, which can be unsubstituted or optionally substituted with up to 2 groups, which can be the same or different, and are selected from alkyl, —O-alkyl and halo.

In another embodiment, for the Compounds of Formula (Ia), R^{1a} is pyridazinyl, which can be unsubstituted or substituted with an alkyl group.

In yet another embodiment, for the Compounds of Formula (Ia), R^{1a} is pyrazinyl, which can be unsubstituted or substituted with an alkyl group.

In another embodiment, for the Compounds of Formula (Ia), R^{1a} is phenyl, which can be unsubstituted or substituted with a —CN group.

In another embodiment, for the Compounds of Formula (Ia), R^{1a} is oxadiazolyl, which can be the same or different, and are selected from alkyl, —O-alkyl, —CN, halo and —C(O)O-alkyl.

In a further embodiment, for the Compounds of Formula (Ia), R^{1a} is selected from:

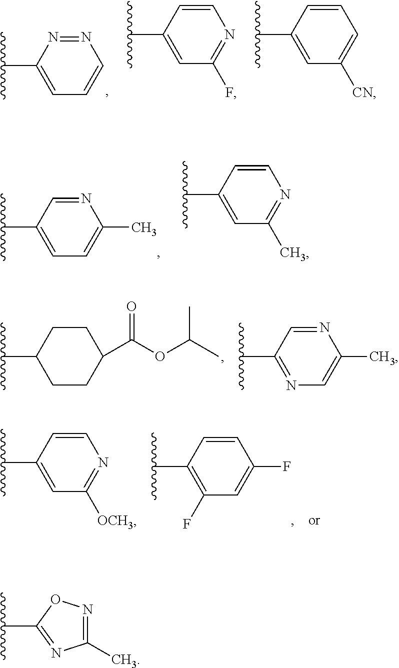

In one embodiment, for the Compounds of Formula (Ia), R^{2a} represents one —F substituent.

In another embodiment, for the Compounds of Formula (Ia), R^{2a} represents two —F substituents.

In another embodiment, for the Compounds of Formula (Ia), R^{2a} represents three —F substituents.

In one embodiment, for the Compounds of Formula (Ia), the ring containing Y and Z has the structure:

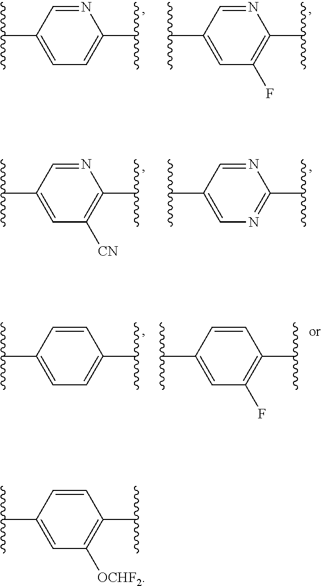

and the ring substituted with R^{2a} has the structure:

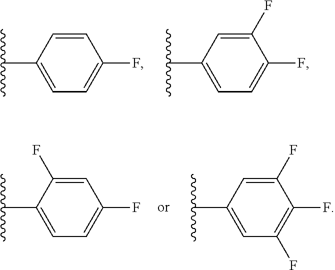

In another embodiment, for the Compounds of Formula (Ia), R^{1a} is selected from:

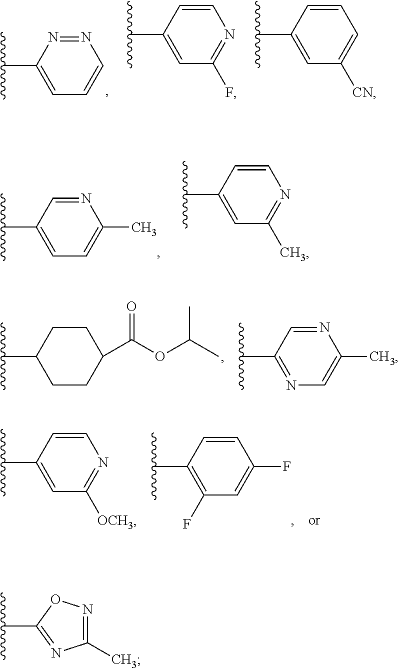

the ring containing Y and Z has the structure:

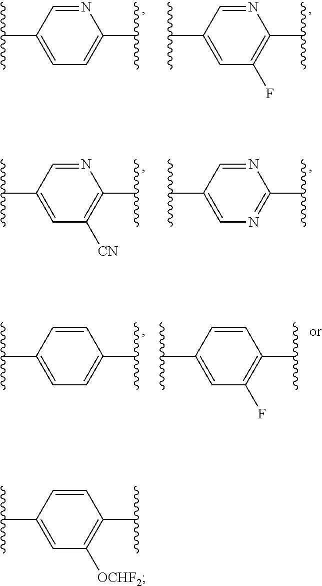

and the ring substituted with R^{2a} has the structure:

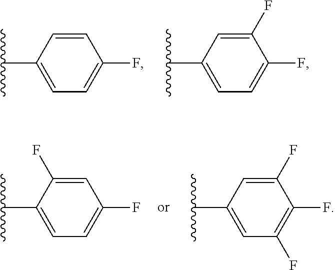

In another embodiment, for the Compounds of Formula (Ia), R^{1a} is methoxy or —OCHF₂; the ring containing Y and Z has the structure:

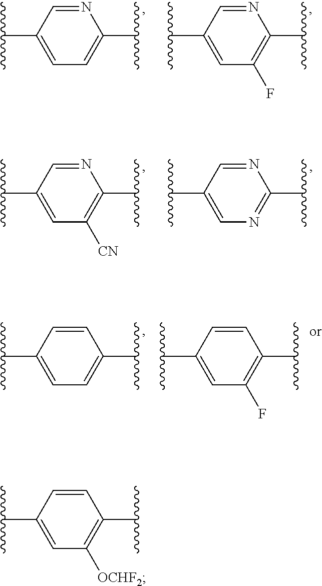

and
the ring substituted with R^{2a} has the structure:

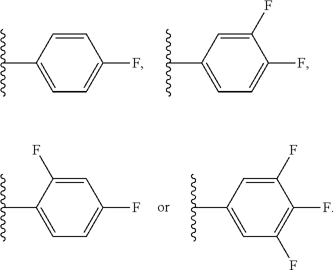

In one embodiment, for the Compounds of Formula (Ia), variables Y, Z, R^{1a} and R^{2a} are selected independently of each other.

In another embodiment, a Compound of Formula (Ia) is in purified form.

In another embodiment, a Compound of Formula (Ia) can be an antagonist of P2X₇.

Non-limiting examples of the Compounds of Formula (I) include the following compounds:

| No. | Structure |
|---|---|
| 1 | 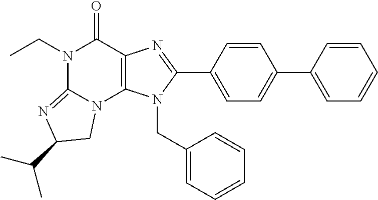 |
| 2 | 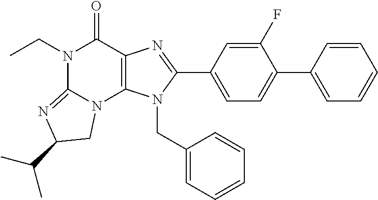 |
| 3 | 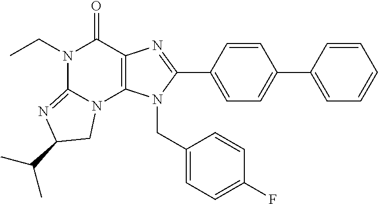 |
| 4 | 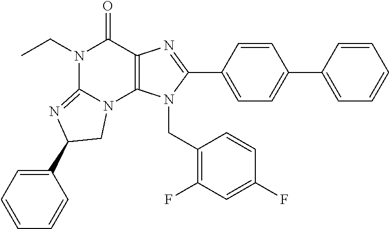 |
| 5 | 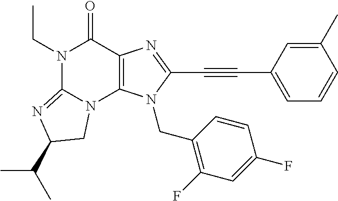 |
| 6 | 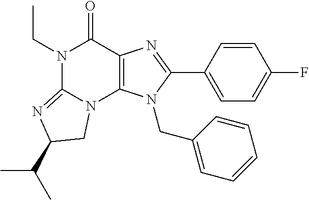 |
| 7 | 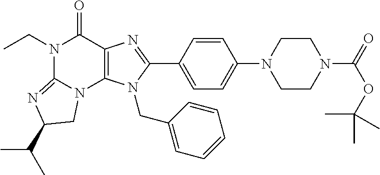 |
| 8 | 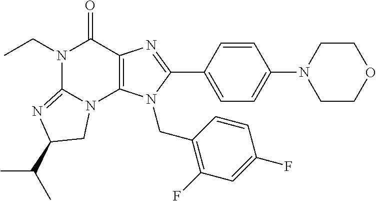 |
| 9 | 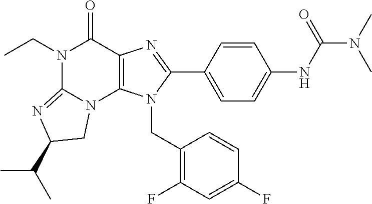 |
| 10 | 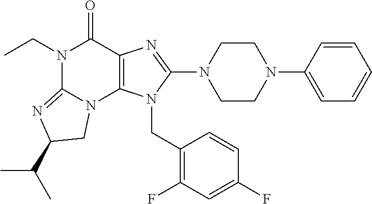 |
| 11 | 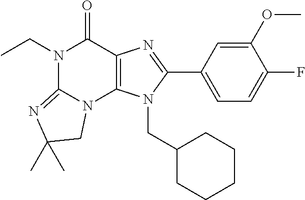 |
| 12 | 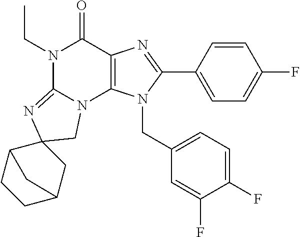 |
| 13 | 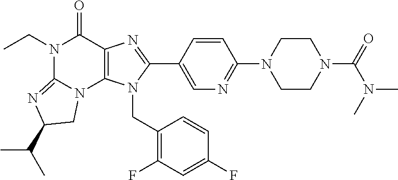 |
| 14 | 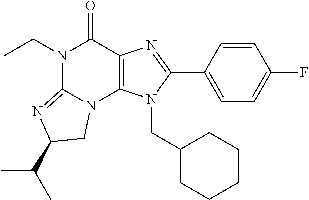 |
| 15 | 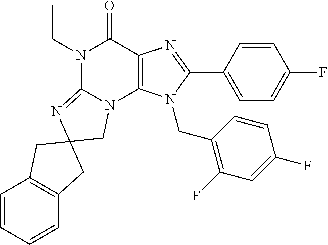 |
| 16 | 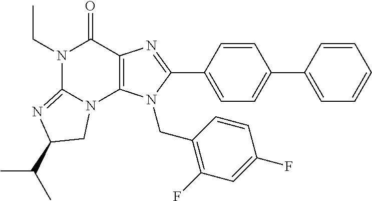 |
| 17 | 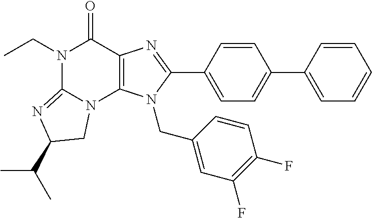 |
| 18 | 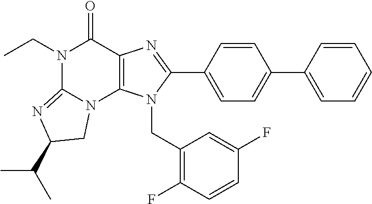 |
| 19 | 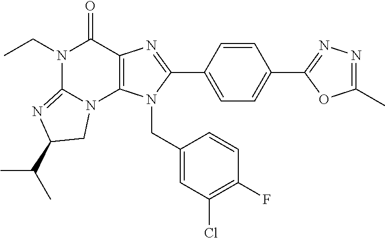 |
| 20 | 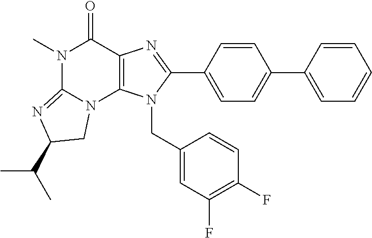 |
| 21 | 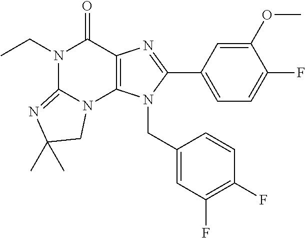 |
| 22 | 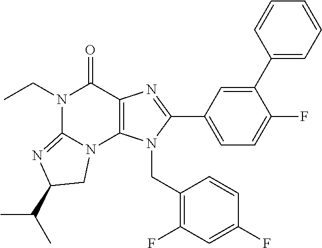 |
| 23 | 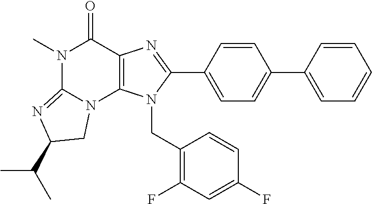 |
| 24 | 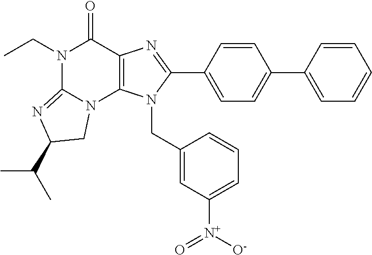 |
| 25 | 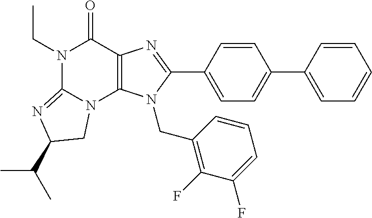 |
| 26 | 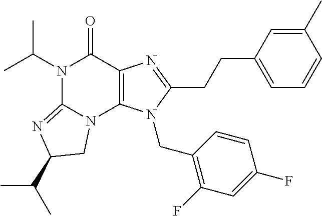 |
| 27 | 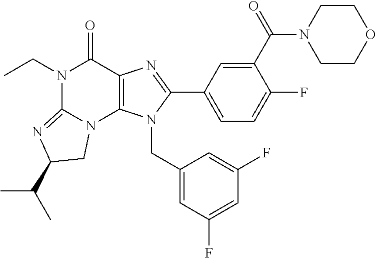 |
| 28 | 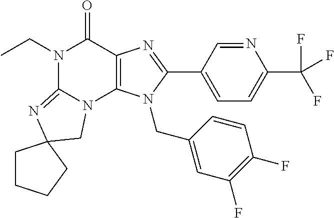 |
| 29 | 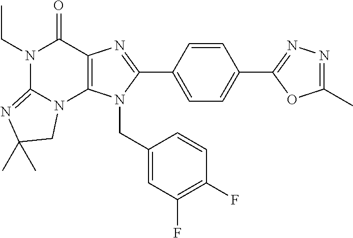 |
| 30 | 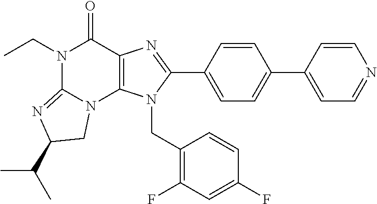 |
| 31 | 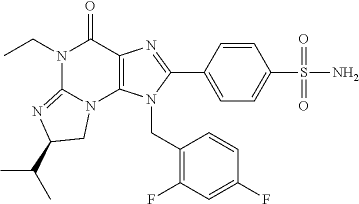 |
| 32 | 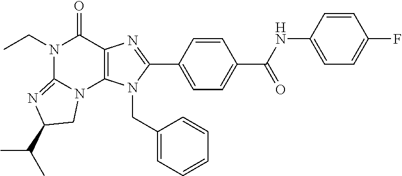 |
| 33 | 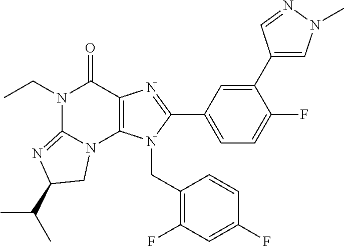 |
| 34 | 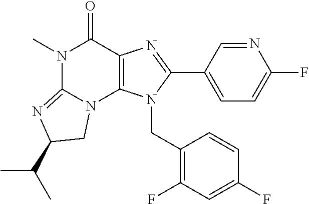 |
| 35 | 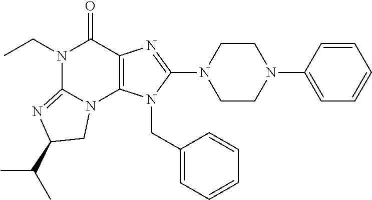 |
| 36 | 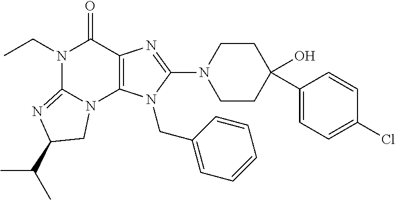 |
| 37 | 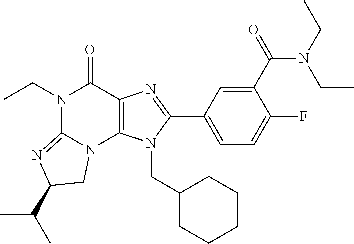 |
| 38 | 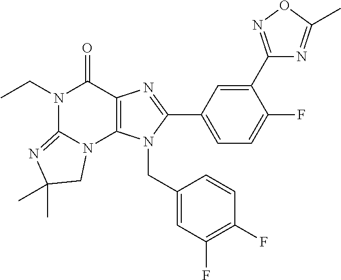 |
| 39 | 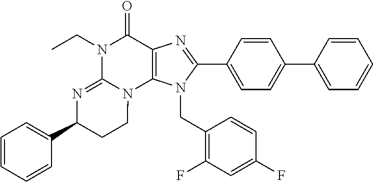 |
| 40 | 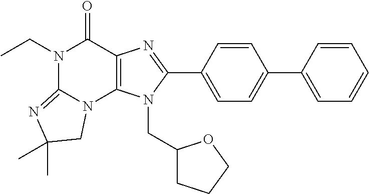 |
| 41 | 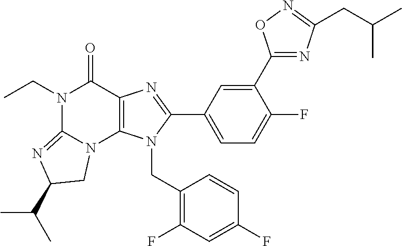 |
| 42 | 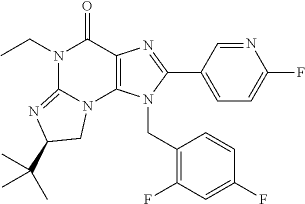 |
| 43 | 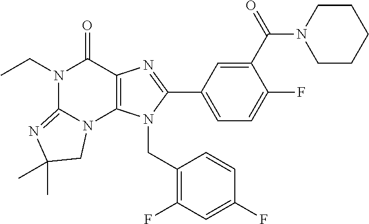 |
| 44 | 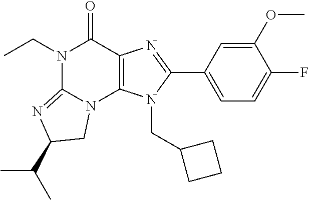 |
| 45 | 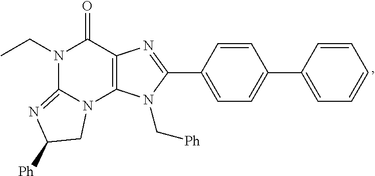 |
| 46 | 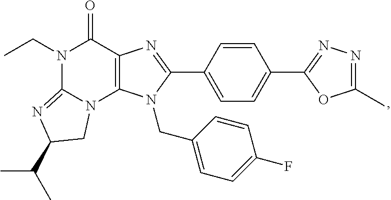 |
| 47 | 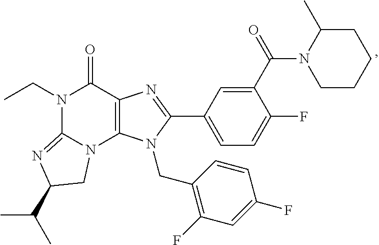 |
| 48 | 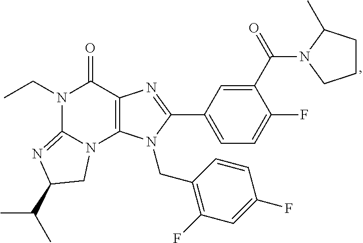 |
| 49 | 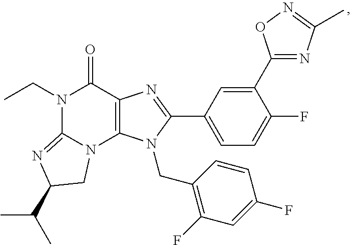 |
| 50 | 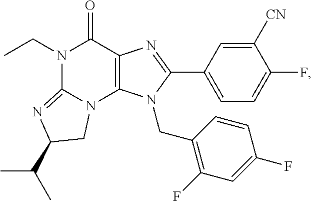 |
| 51 | 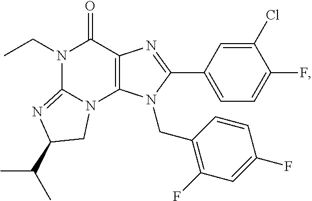 |
| 52 | 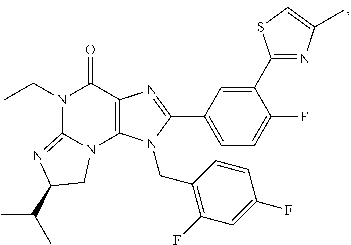 |
| 53 | 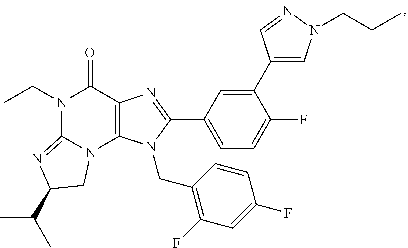 |
| 54 | 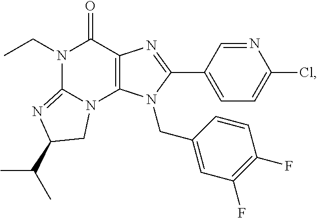 |
| 55 | 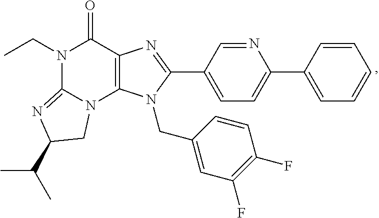 |
| 56 | 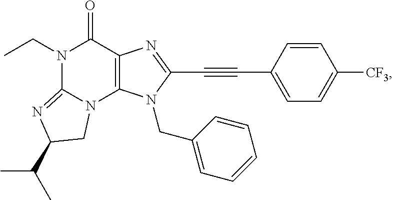 |
| 57 | 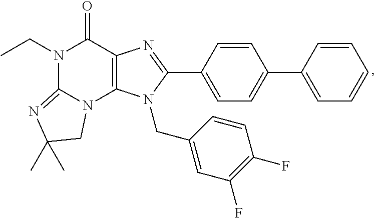 |
| 58 | 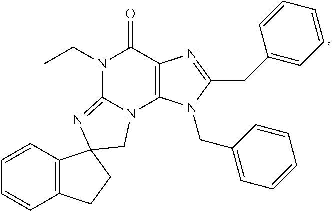 |
| 59 | 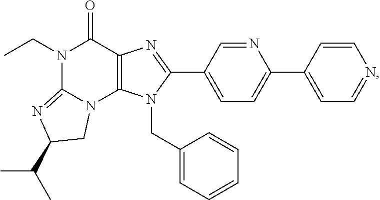 |
| 60 | 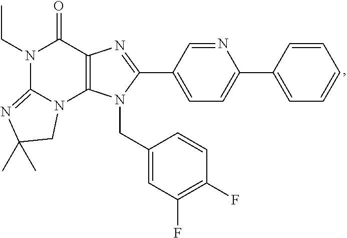 |
| 61 | 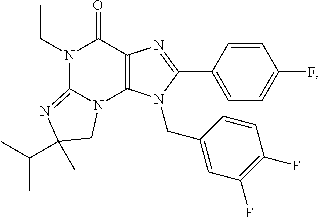 |
| 62 | 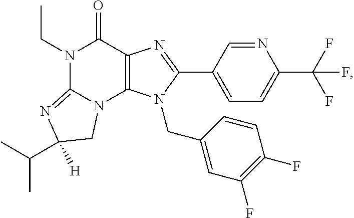 |
| 63 | 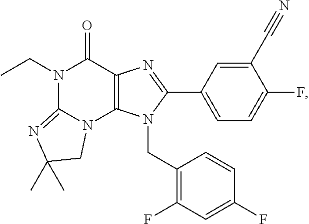 |
| 64 | 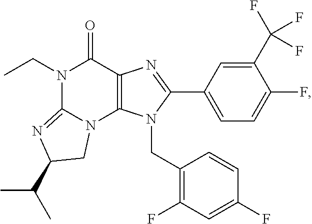 |
| 65 | 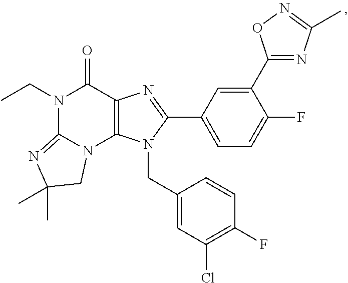 |
| 66 | 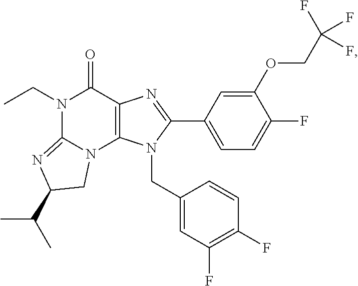 |
| 67 | 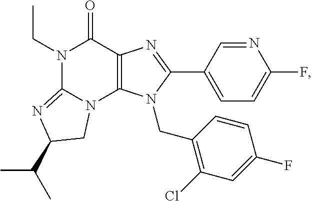 |
| 68 | 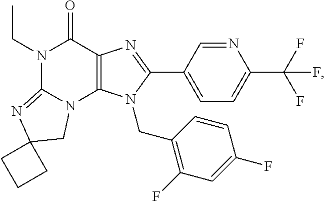 |
| 69 | 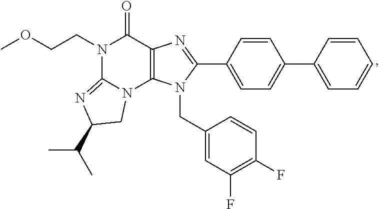 |
| 70 | 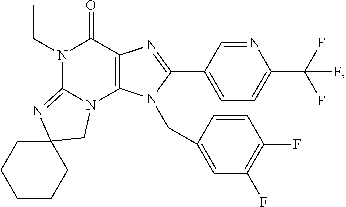 |
| 71 | 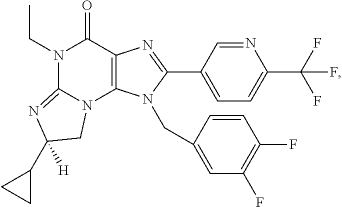 |
| 72 | 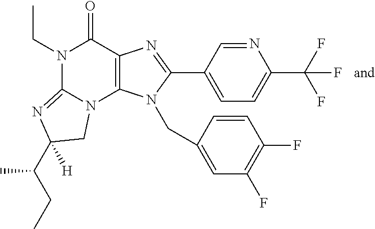 |
| 73 | 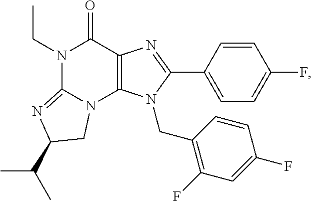 |

and pharmaceutically acceptable salts, solvates, esters and prodrugs thereof.

Additional illustrative examples of the Compounds of Formula (I) include, but are not limited to, the following compounds:

| No. | Structure |
|---|---|
| 74 | 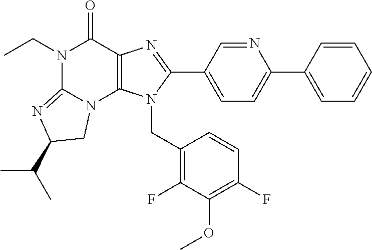 |
| 75 | 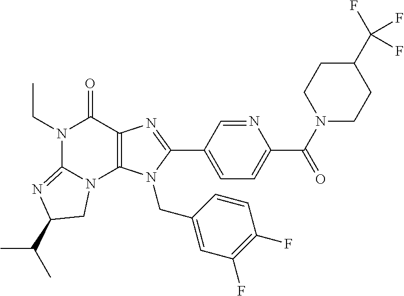 |
| 76 | 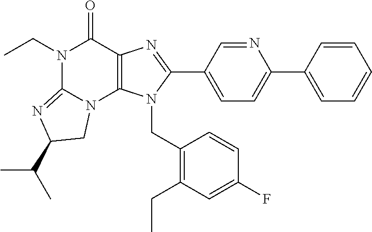 |
| 77 | 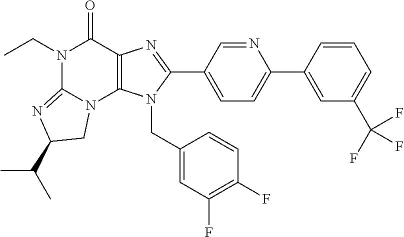 |
| 78 | 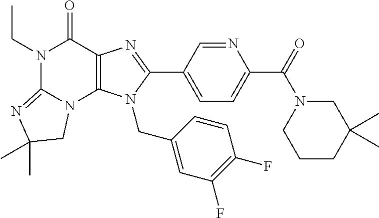 |
| 79 | 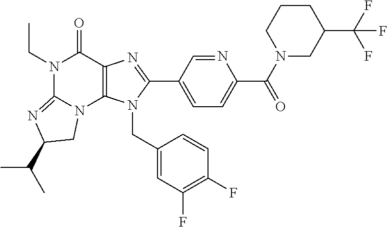 |
| 80 | 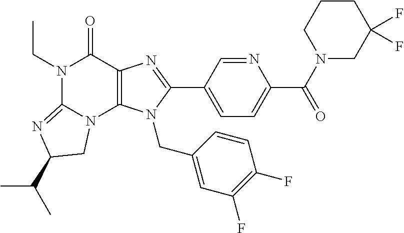 |
| 81 | 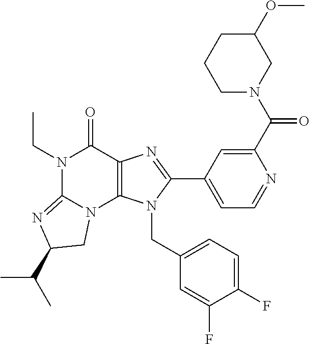 |
| 82 | 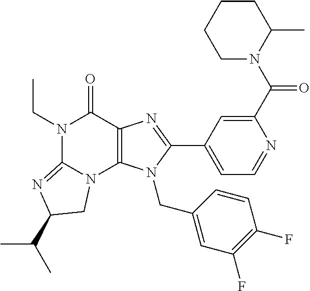 |
| 83 | 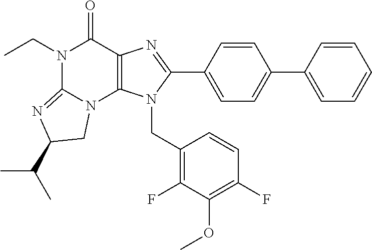 |
| 84 | 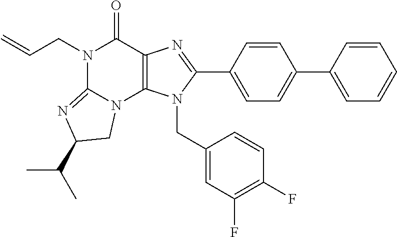 |
| 85 | 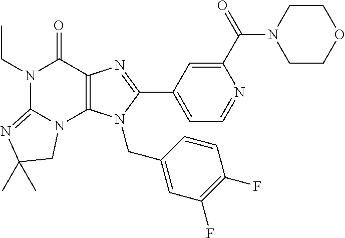 |
| 86 | 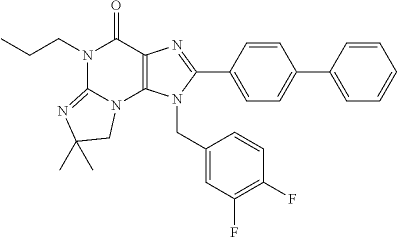 |
| 87 | 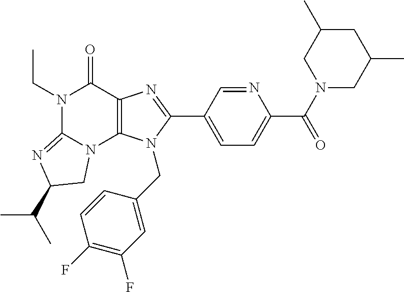 |
| 88 | 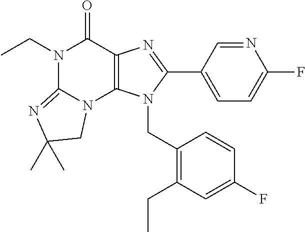 |
| 89 | 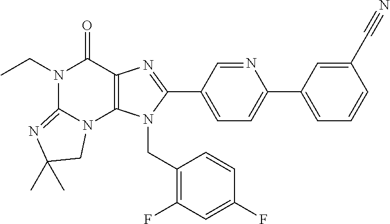 |
| 90 | 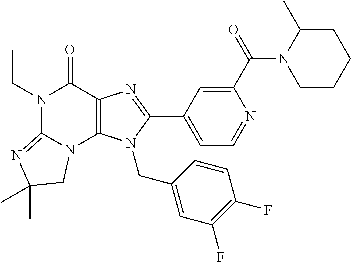 |
| 91 | 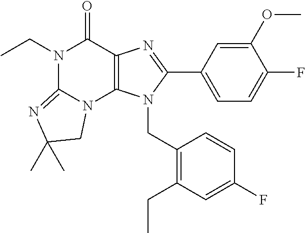 |
| 92 | 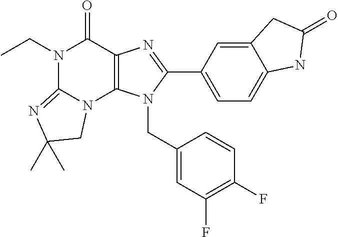 |
| 93 | 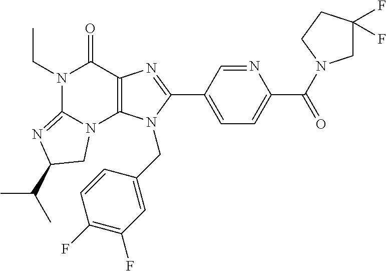 |
| 94 | 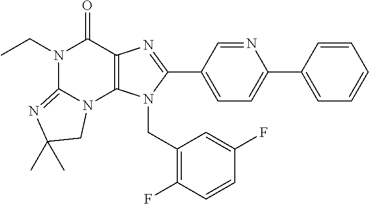 |
| 95 | 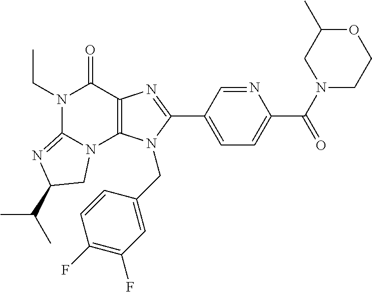 |
| 96 | 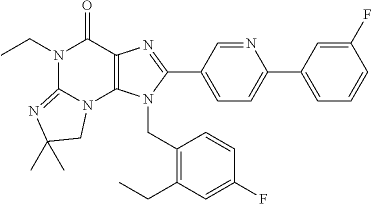 |
| 97 | 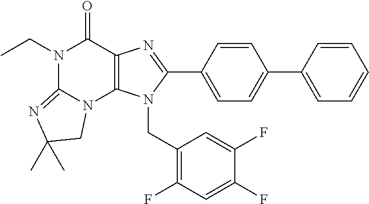 |
| 98 | 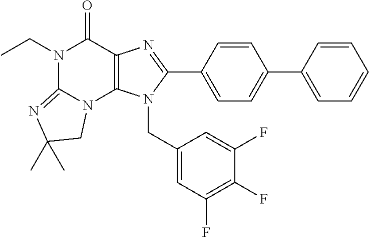 |
| 99 | 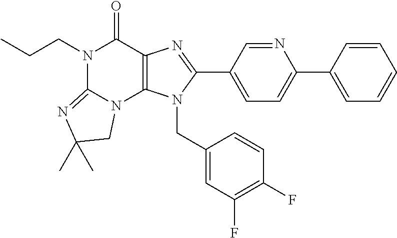 |
| 100 | 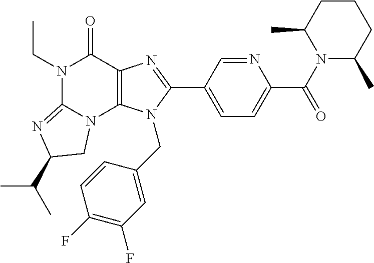 |
| 101 | 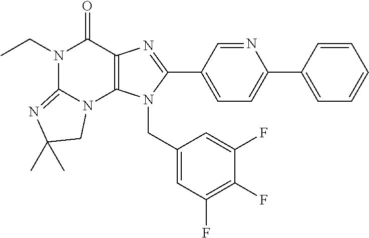 |
| 102 | 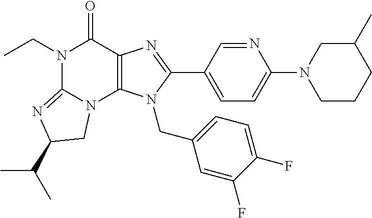 |
| 103 | 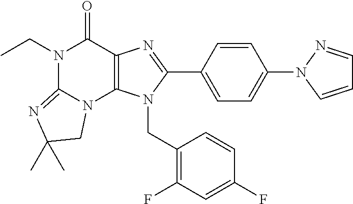 |
| 104 | 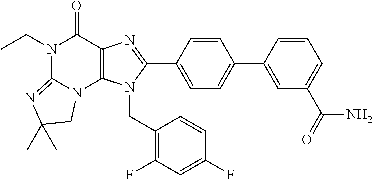 |
| 105 | 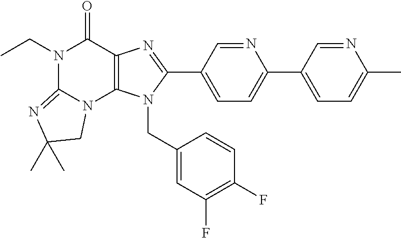 |
| 106 | 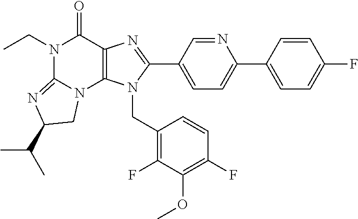 |
| 107 | 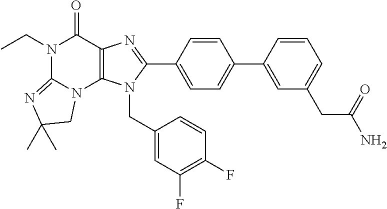 |
| 108 | 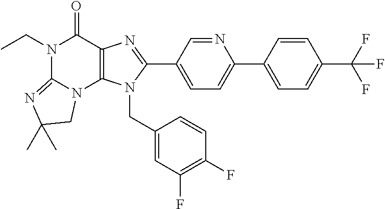 |
| 109 | 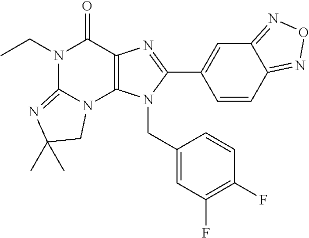 |
| 110 | 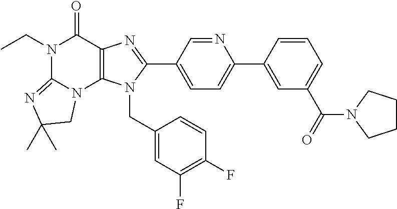 |
| 111 | 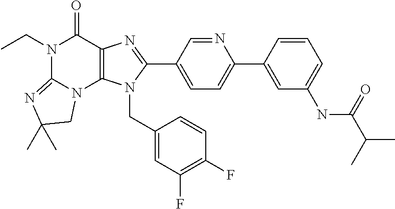 |
| 112 | 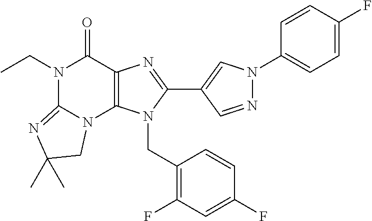 |
| 113 | 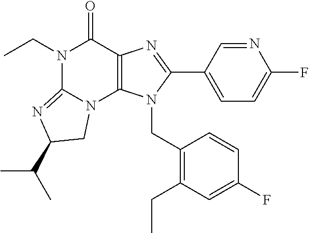 |
| 114 | 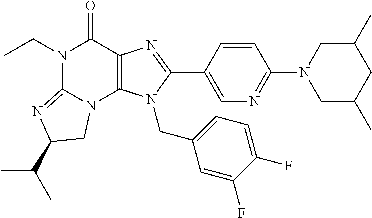 |
| 115 | 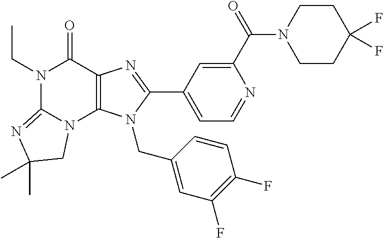 |
| 116 | 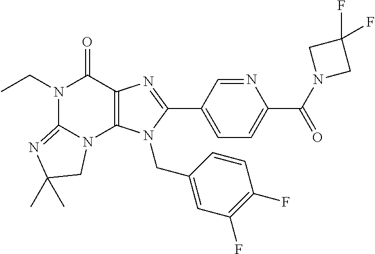 |
| 117 | 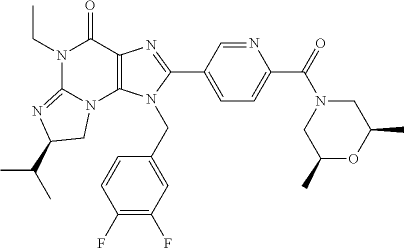 |
| 118 | 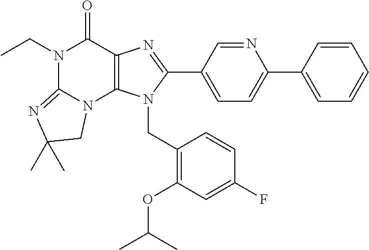 |
| 119 | 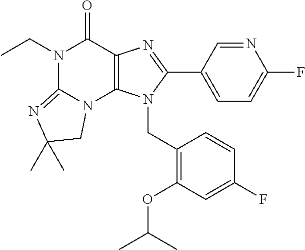 |
| 120 | 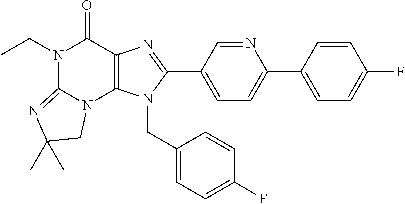 |
| 121 | 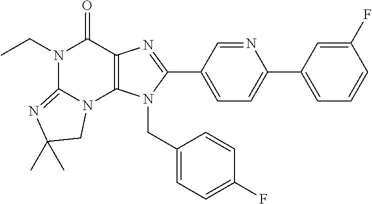 |
| 122 | 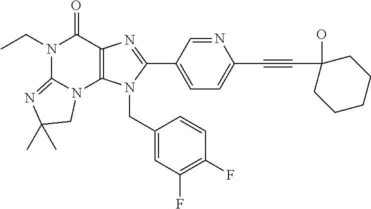 |
| 123 | 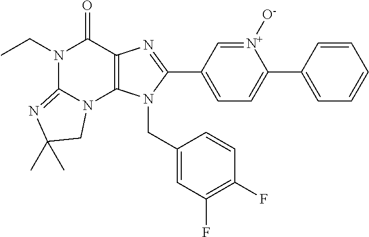 |
| 124 | 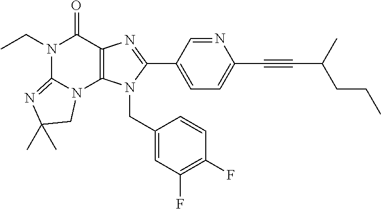 |
| 125 | 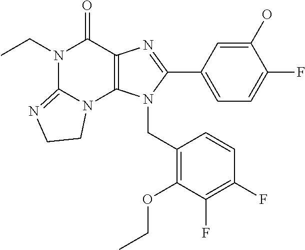 |
| 126 | 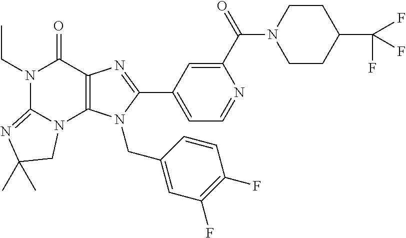 |
| 127 | 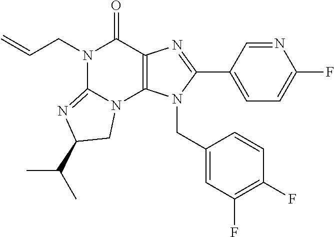 |
| 128 | 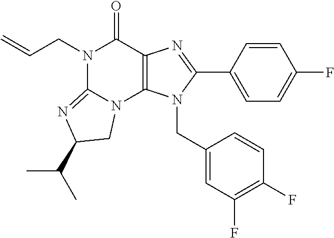 |
| 129 | 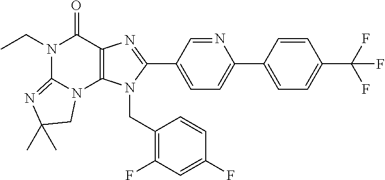 |
| 130 | 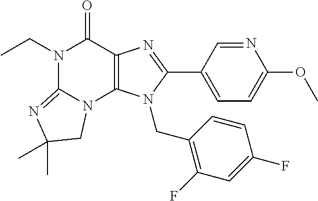 |
| 131 | 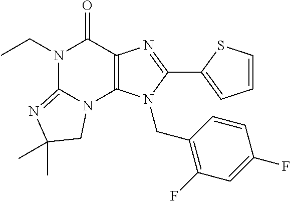 |
| 132 | 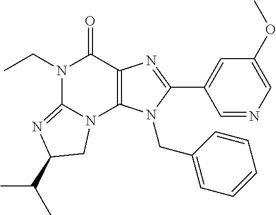 |
| 133 | 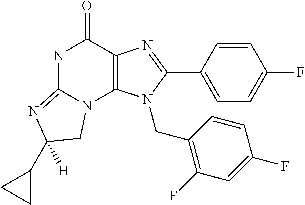 |
| 134 | 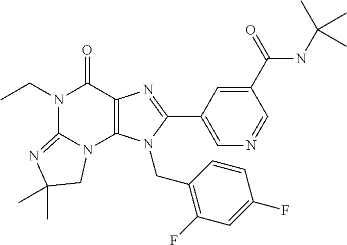 |
| 135 | 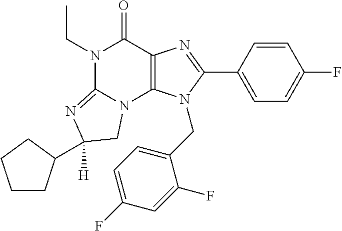 |
| 136 | 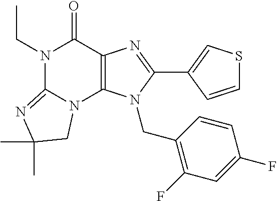 |
| 137 | 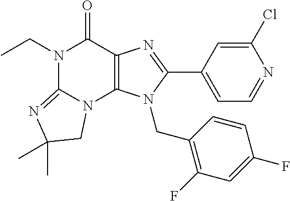 |
| 138 | 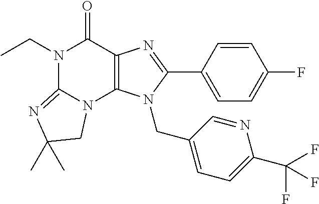 |
| 139 | 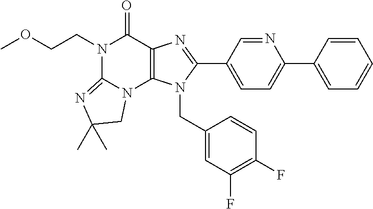 |
| 140 | 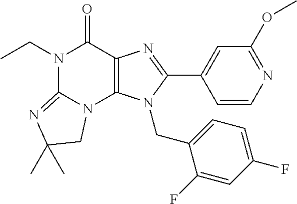 |
| 141 | 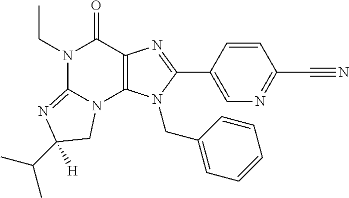 |
| 142 | 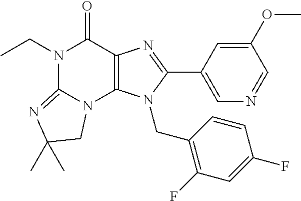 |
| 143 | 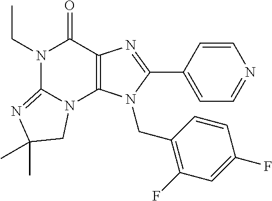 |
| 144 | 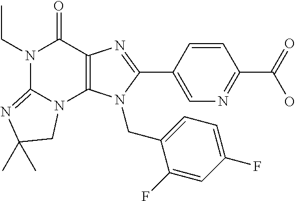 |
| 145 | 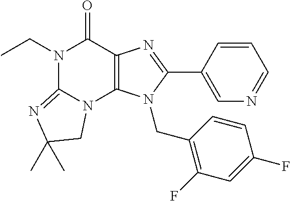 |
| 146 | 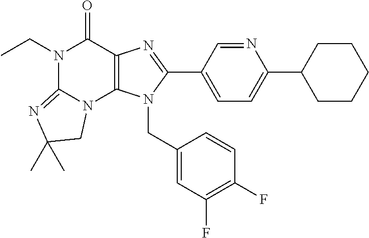 |
| 147 | 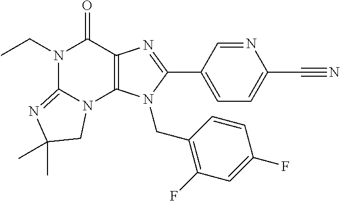 |
| 148 | 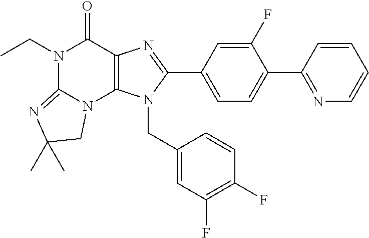 |
| 149 | 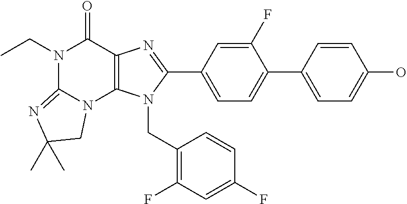 |
| 150 | 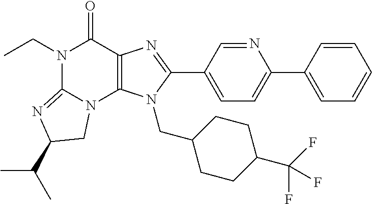 |
| 151 | 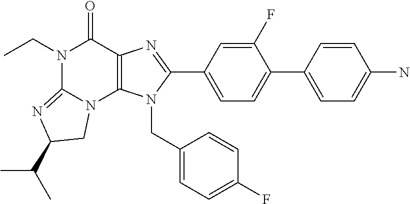 |
| 152 | 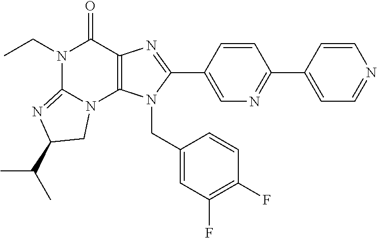 |
| 153 | 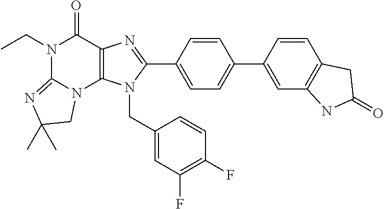 |
| 154 | 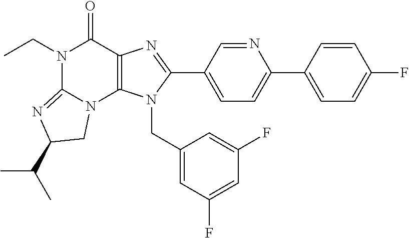 |
| 155 | 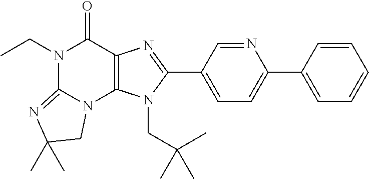 |
| 156 | 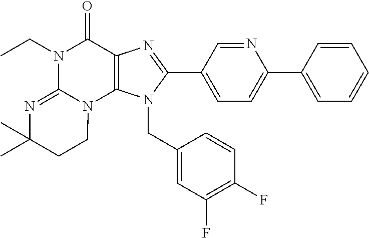 |
| 157 | 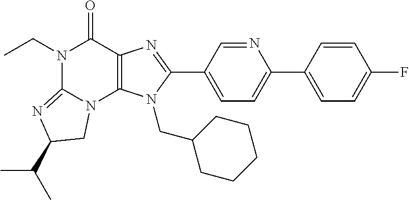 |
| 158 | 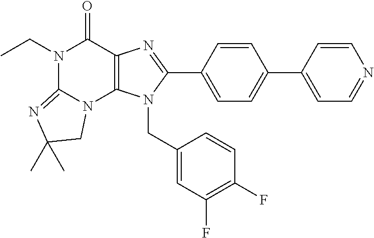 |
| 159 | 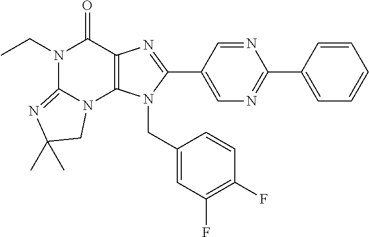 |
| 160 | 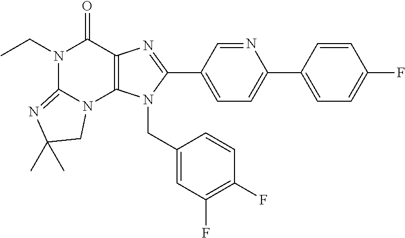 |

and pharmaceutically acceptable salts, solvates, esters and prodrugs thereof.

Non-limiting illustrative examples of the Compounds of Formula (Ia) include, but are not limited to, the following compounds:

| No. | Structure |
|---|---|
| 161 | 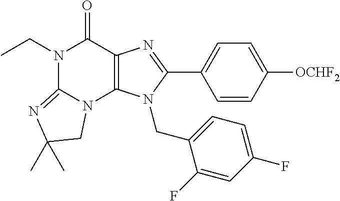 |
| 162 | 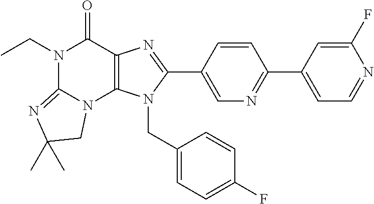 |
| 163 | 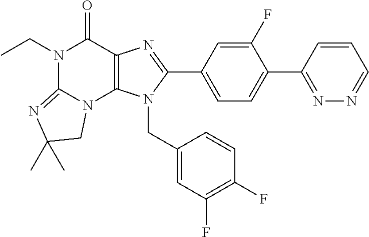 |
| 164 | 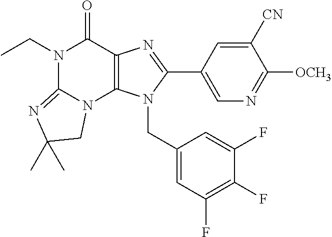 |
| 165 | 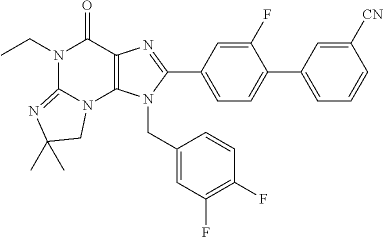 |
| 166 | 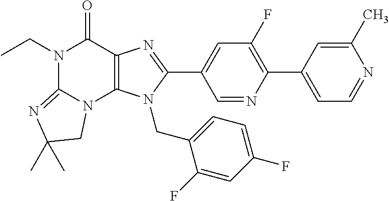 |
| 167 | 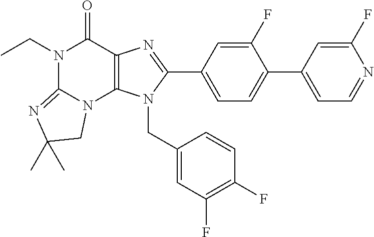 |
| 168 | 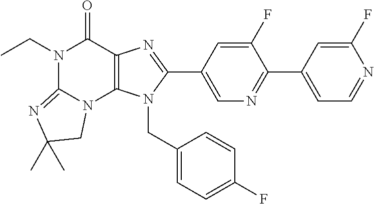 |
| 169 | 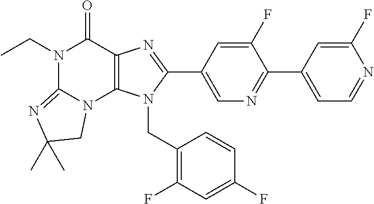 |
| 170 | 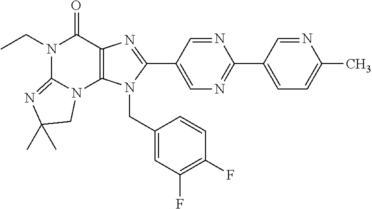 |
| 171 | 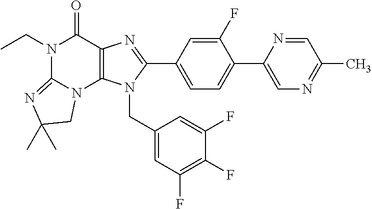 |
| 172 | 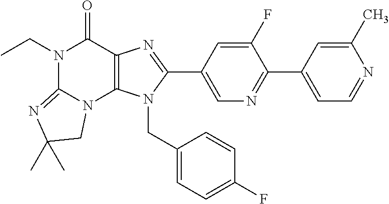 |
| 173 | 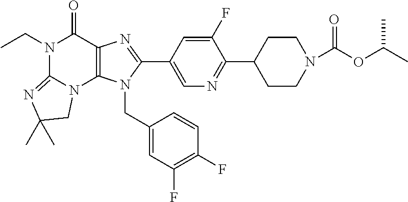 |
| 174 | 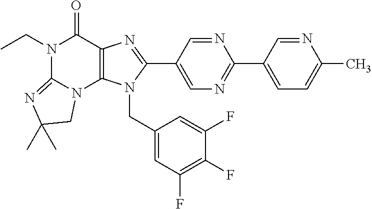 |
| 175 | 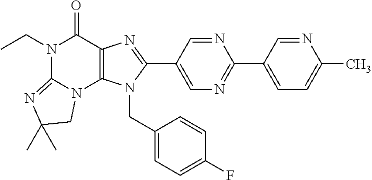 |
| 176 | 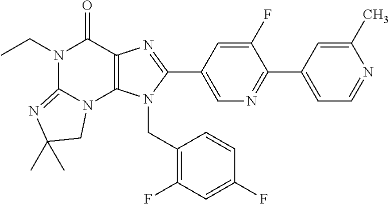 |
| 177 | 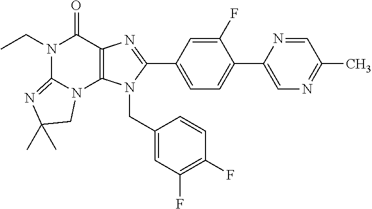 |
| 178 | 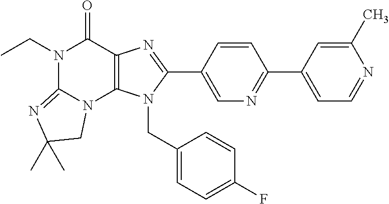 |
| 179 | 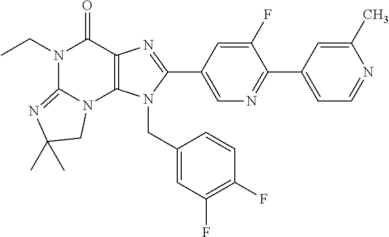 |
| 180 | 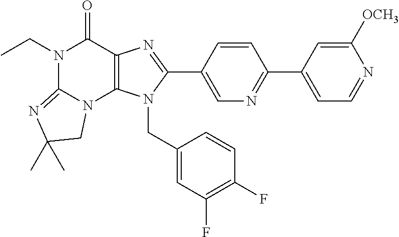 |
| 181 | 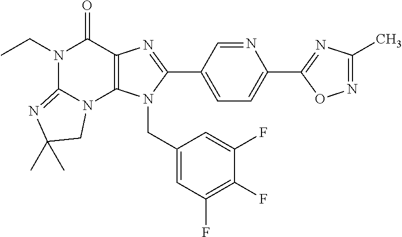 |
| 182 | 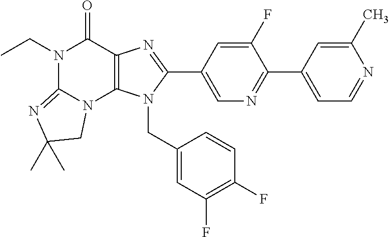 |
| 183 | 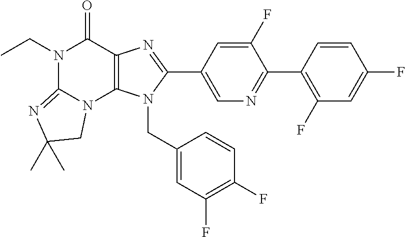 |
| 184 | 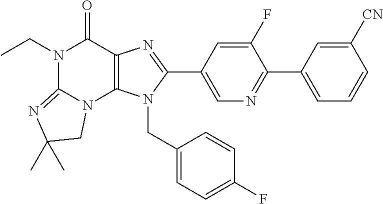 |
| 185 | 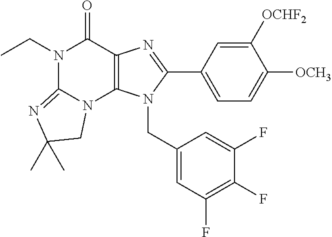 |

and pharmaceutically acceptable salts, solvates, esters and prodrugs thereof.

### Methods For Making the Compounds of Formula (I)

Methods useful for making the Compounds of Formula (I) are set forth in the Examples below and generalized in Schemes 1-5. Alternative synthetic pathways and analogous structures will be apparent to those skilled in the art of organic synthesis. Alternatively, the Compounds of Formula (I) may also be made using methods described in U.S. Pat. No. 5,393,755.

Scheme 1 illustrates a method useful for making Compounds of Formula iv, which are useful intermediates for making the Compounds of Formula (I), wherein R³ is —CH₂-aryl or —CH₂-heteroaryl.

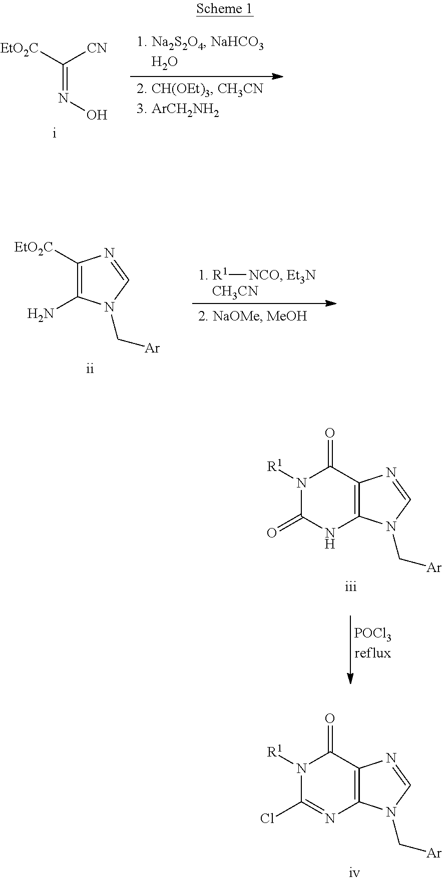

wherein Ar is aryl or heteroaryl.

Ethyl cyanoglyoxylate-2-oxime (i) can be reacted with sodium dithionate in the presence of a base such as NaHCO₃ and the resulting product refluxed with an appropriate substituted benzimidic acid ethyl ester hydrochloride, followed by treatment with an appropriate benzylamine to provide the imidazole Compounds of Formula ii. A Compound of Formula II can then be reacted with an isocynate of formula R¹—NCO in the presence of triethylamine, followed by treatment with methanol to provide the Compounds of Formula iii. A Compound of Formula iii is then reacted with phosphorus oxychloride to provide the Compounds of Formula iv.

Scheme 2 illustrates a method useful for making Compounds of Formula vi, which are useful intermediates for making the Compounds of Formula (I), wherein R³ is —CH₂-aryl or —CH₂-heteroaryl.

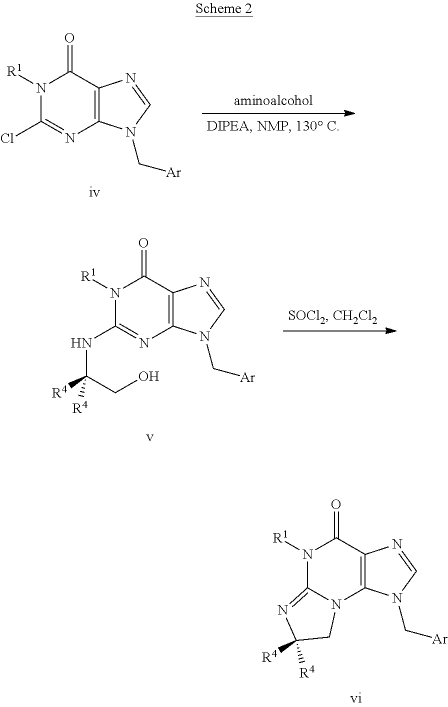

wherein R¹ and R⁴ are defined above for the Compounds of Formula (I) and Ar is aryl or heteroaryl.

A Compound of Formula iv can be coupled with an aminoalcohol in the presence of a non-nucleophilic base, such as DIPEA, to provide the hydroxy Compounds of Formula v. A Compound of Formula v can then be cyclized upon treatment with thionyl chloride to provide the Compounds of Formula vi.

Scheme 3 illustrates a method useful for making the Compounds of Formula ix, which correspond to the PDG's wherein R² is aryl and R³ is —CH₂-aryl or —CH₂-heteroaryl.

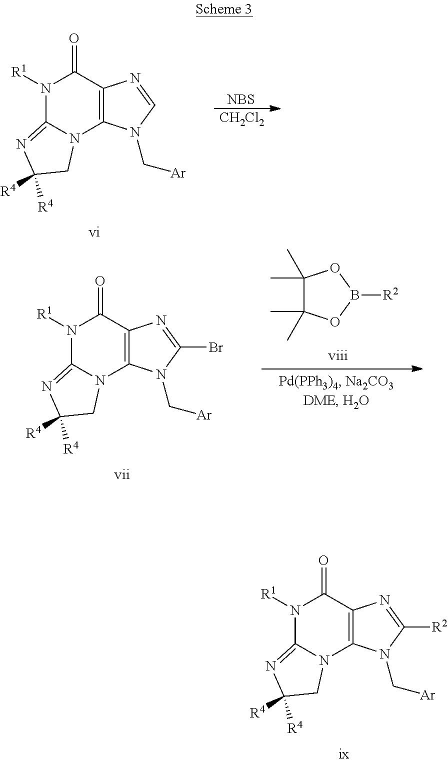

wherein R¹ and R⁴ are defined above for the Compounds of Formula (I), R² is aryl, and Ar is aryl or heteroaryl.

A Compound of Formula vi can be brominated using N-bromosuccinimide to provide the bromo Compounds of Formula vii, which can subsequently be coupled with a boronic ester of formula viii via a Suzuki coupling reaction to provide the Compounds of Formula ix. The boronic acid esters of formula viii can be made using the methods set forth in the examples section herein or via methods well-known to those of ordinary skill in the art of organic synthesis.

Scheme 4 shows an alternative method for making the Compounds of Formula (I).

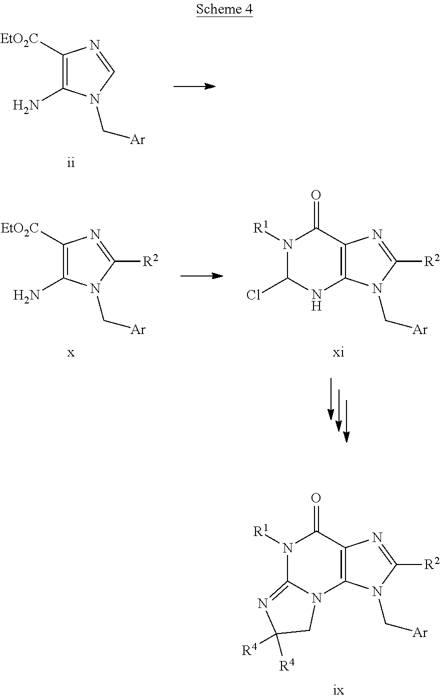

wherein R¹ and R⁴ are defined above for the Compounds of Formula (I), R² is aryl, and Ar is aryl or heteroaryl.

An imidazole Compound of Formula II can be brominated then coupled with a boronic acid ester to provide a Compound of Formula x using methodology set forth in Scheme 3 above. A Compound of Formula x can then be cyclized to provide the bicyclic Compounds of Formula xi using methodology set forth in Scheme 1 above. A Compound of Formula xi is then cyclized using the method set forth in Scheme 2 above to provide the bicyclic Compounds of Formula ix.

Scheme 5 illustrates an alternative method for making the Compounds of Formula (I).

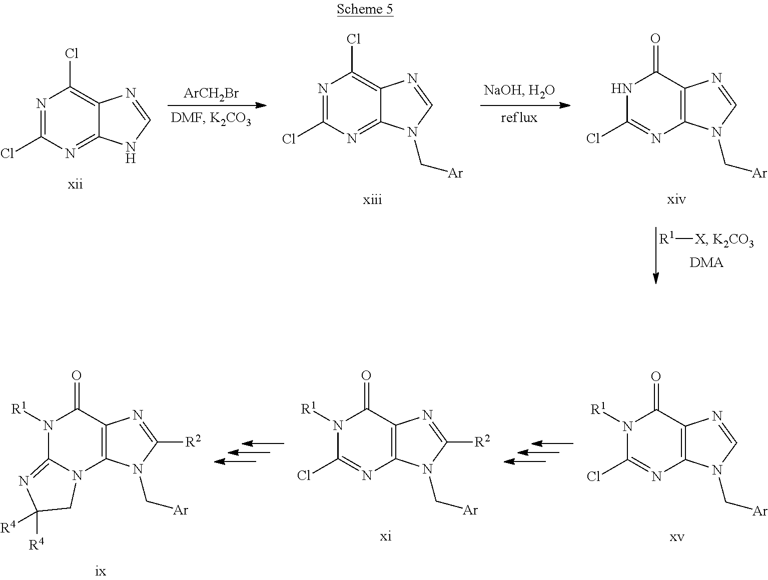

wherein R¹ and R⁴ are defined above for the Compounds of Formula (I), R² is aryl, Ar is aryl or heteroaryl, and X is a good leaving group, such as —Br, —Cl, —I, —O-mesyl, —O-tosyl or —O-triflyl.

The commercially available Compound of Formula xii can be reacted with an arylalkyl bromide in the presence of a base, such as potassium carbonate, to provide the N-derivatized Compounds of Formula xiii. A Compound of Formula xiii can then be converted to the Compounds of Formula xiv upon exposure to a hydroxide base, such as sodium hydroxide. A Compound of Formula xiv can then be reacted with a Compound of Formula R¹X in the presence of a carbonate base, such as potassium carbonate to provide the Compounds of Formula xv. The Compounds of Formula xv can finally be converted to the Compounds of Formula xi as described above in Scheme 4.

The starting materials and reagents depicted in Schemes 1-5 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art of organic synthesis will recognize that the preparation of the Compounds of Formula (I) may require the need for the protection of certain functional groups (i.e., derivatization for the purpose of chemical compatibility with a particular reaction condition). Suitable protecting groups for the various functional groups of the Compounds of Formula (I) and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

Solvents, reagents, and intermediates that are commercially available were used as received unless otherwise indicated. Reagents and intermediates that are not commercially available were prepared in the manner described below. ¹H NMR spectra were obtained on a Varian 400 MHz or Balker 300 MHz instrument and are reported as ppm down field from Me₄Si with number of protons, multiplicities, and coupling constants in Hertz indicated parenthetically.

### Example 1

### Preparation of Compound 2a

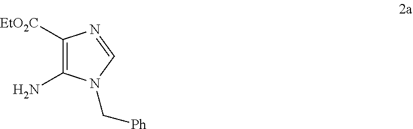

A 2-L three-necked round-bottomed flask equipped with a mechanical stirrer was charged with ethyl cyanoglyoxylate-2-oxime (40.0 g, 0.281 mol) and saturated aqueous NaHCO₃ (300 mL). With vigorous stirring, water (300 mL) was added, followed by portionwise addition of sodium dithionite (200 g, 1.15 mol) over 45 minutes. After stirring for 15 minutes, the solution was extracted with CH₂Cl₂ (4×250 mL) and the combined organic extracts were dried over sodium sulfate, filtered and concentrated in vacuo to provide a yellow oil. To a stirred solution of this yellow oil in CH₃CN (150 mL) was added triethyl orthoformate (42.6 mL, 0.256 mol). The reaction mixture was heated to reflux and allowed to stir at this temperature for 30 minutes, then cooled to room temperature. Benzylamine (31.1 mL, 0.285 mol) was then added and the resulting reaction was heated to reflux and allowed to stir at this temperature for 30 minutes, then cooled to room temperature and concentrated in vacuo. The brown viscous oil was triturated with EtOAc (150 mL) and the resulting suspension was stirred overnight. The precipitate was collected, washed with cold EtOAc (2×100 mL), air-dried for 1 hour and further dried in a vacuum oven at 50° C. to provide compound 2a (23.7 g, 34%) as an-off-white solid.

### Example 2

### Preparation of Compound 2b

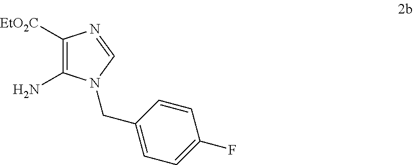

Using the method described in Example 1 and substituting 4-fluorobenzylamine for benzylamine, compound 2b was synthesized as an off-white solid.

### Example 3

### Preparation of Compound 2c

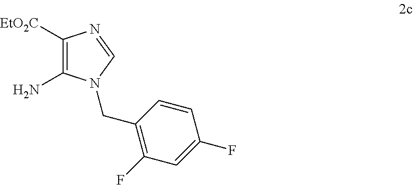

Using the method described in Example 1 and substituting 2,4-difluorobenzylamine for benzylamine, compound 2b was synthesized as an off-white solid.

### Example 4

### Preparation of Compound 2d

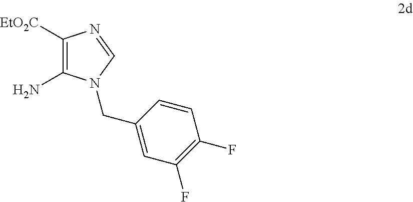

Using the method described in Example 1 and substituting 3,4-difluorobenzylamine for benzylamine, compound 2b was synthesized as an off-white solid.

### Example 5

### Preparation of Compound 2e

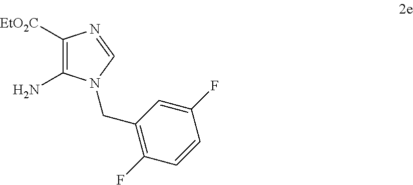

Using the method described in Example 1 and substituting 2,5-difluorobenzylamine for benzylamine, compound 2b was synthesized as an off-white solid.

### Example 6

### Preparation of Compound 3a

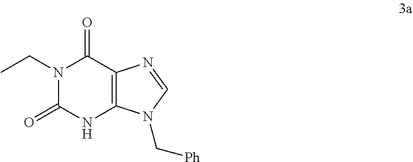

A 2-L three-necked round-bottomed flask equipped with a magnetic stir bar was charged with ethyl 5-amino-1-benzyl-1H-imidazole-4-carboxylate (2a) (50.0 g, 0.204 mol), ethyl isocyanate (64 mL, 0.816 mol), triethylamine (142 ml, 1.02 mol) and toluene (450 mL). The reaction mixture was heated under reflux for 18 hours, cooled to room temperature and concentrated in vacuo to a black solid. To a mechanical stirred solution of this solid in MeOH (600 mL) was added sodium methoxide (55.1 g, 1.02 mol) portionwise over 5 minutes. The reaction mixture was heated under reflux for 1 hour, cooled to room temperature, concentrated in vacuo and diluted with water (600 mL). The aqueous mixture was washed with CH₂Cl₂ (3×1 L) and acidified to pH 6 using 2 N HCl (475 mL). The resulting precipitate was filtered and the filter cake washed with water (3×250 mL) and diethyl ether (2×200 mL). The solid was dried in a vacuum oven at 45° C. for 18 hours to provide compound 3a (38.7 g, 70%) as a light brown solid.

### Example 7

### Preparation of Compound 3b

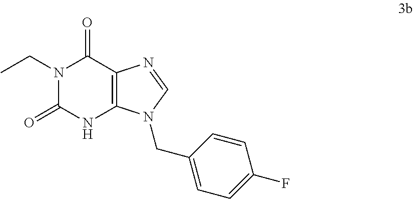

Using the method described in Example 6 and substituting compound 2b for compound 2a, compound 3b was synthesized as an off-white solid.

### Example 8

### Preparation of Compound 3c

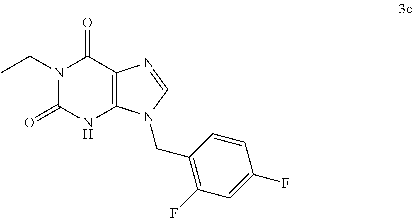

Using the method described in Example 6 and substituting compound 2c for compound 2a, compound 3c was synthesized as an off-white solid.

### Example 9

### Preparation of Compound 3d

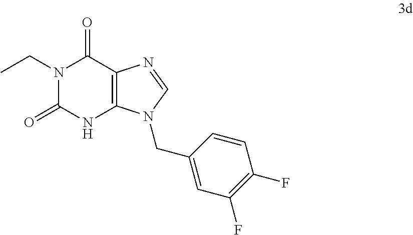

Using the method described in Example 6 and substituting compound 2d for compound 2a, compound 3d was synthesized as an off-white solid.

### Example 10

### Preparation of Compound 3e

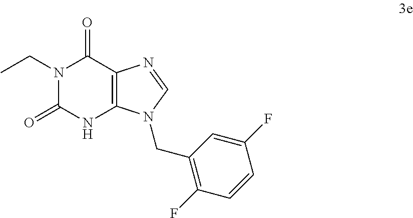

Using the method described in Example 6 and substituting compound 2e for compound 2a, compound 3e was synthesized as an off-white solid.

### Example 11

### Preparation of Compound 4a

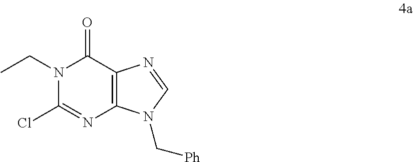

A 1-L three-necked round-bottomed flask equipped with a mechanical stirrer was charged with 9-benzyl-1-ethyl-1H-purine-2,6(3H,9H)-dione (3a) (38.7 g, 0.143 mol) and phosphorus oxychloride (387 mL, 4.15 mol). The mixture was heated under reflux for 18 hours, more phosphorus oxychloride (100 mL, 1.07 mol) was added and the reaction was heated to reflux for an additional 4 hours. The reaction mixture was concentrated in vacuo and the resulting brown oil was carefully poured into a mixture of ice/water (1 L) and CH₂Cl₂ (500 mL). After adjustment of the mixture to pH 7-8 using solid NaHCO₃, the layers were separated and the aqueous phase was extracted with CH₂Cl₂ (3×1 L). The combined organic extracts were dried over Na₂SO₄, filtered and concentrated in vacuo. The brown residue was purified using column chromatography (silica gel, 97:3, CH₂Cl₂/MeOH) to provide a yellow solid. This solid was further purified using column chromatography (silica gel, 80:20, EtOAc/hexanes) to provide compound 4a (25.2 g, 61%) as a yellow solid.

### Example 12

### Preparation of Compound 4b

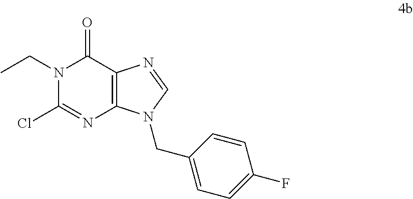

Using the method described in Example 11 and substituting compound 3b for compound 3a, compound 4b was synthesized as a yellow solid.

### Example 13

### Preparation of Compound 4c

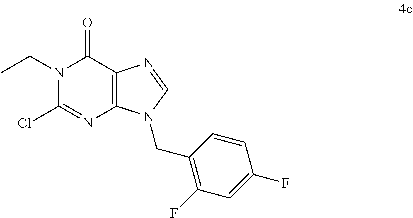

Using the method described in Example 11 and substituting compound 3c for compound 3a, compound 4c was synthesized as a yellow solid.

### Example 14

### Preparation of Compound 4d

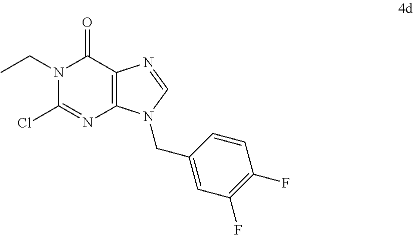

Using the method described in Example 11 and substituting compound 3d for compound 3a, compound 4d was synthesized as a yellow solid.

### Example 15

### Preparation of Compound 4e

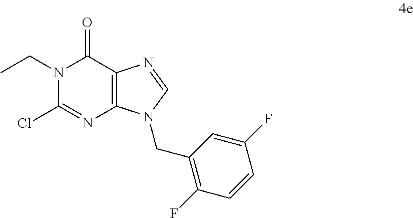

Using the method described in Example 11 and substituting compound 3e for compound 3a, compound 4e was synthesized as a yellow solid.

### Example 16

### Preparation of Compound 6a

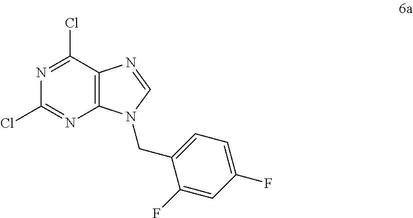

A 2-L three-necked round-bottomed flask equipped with a mechanical stirrer was charged with 2,6-dichloropurine (5) (30.0 g, 0.159 mol), potassium carbonate (110 g, 0.794 mol) and dry DMF (600 mL). The suspension was stirred vigorously as 2,4-difluorobenzyl bromide (39.4 g, 0.190 mol) was added via addition funnel over 0.5 hours. After stirring at room temperature for 18 hours, EtOAc (1 L) and water (1.7 L) was added and the layers separated. The aqueous layer was extracted with EtOAc (2×500 mL) and the combined organic extracts were washed with brine (3×700 mL), dried over Na₂SO₄, filtered and concentrated in vacuo. The resulting residue was triturated with EtOAc (200 mL) and stirred for 15 hours. The precipitate was collected, washed with cold 20% EtOAc in hexanes and air-dried overnight to provide compound 6a (16.6 g, 33%) as a white solid. The filtrate was concentrated in vacuo and the residue obtained was purified using CombiFlash chromatography (silica gel, 50:50, hexanes/EtOAc) to provide additional product 6a (14.4 g, 29%) as a white solid.

### Example 17

### Preparation of Compound 6b

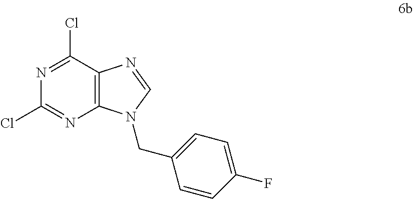

Using the method described in Example 16 and substituting 4-fluorobenzyl bromide for 2,4-difluorobenzyl bromide, compound 6b was synthesized as a white solid.

### Example 18

### Preparation of Compound 6c

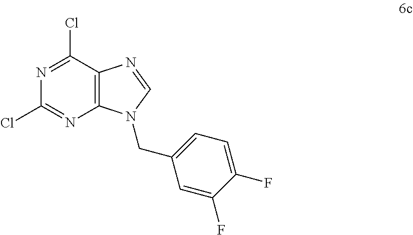

Using the method described in Example 16 and substituting 3,4-difluorobenzyl bromide for 2,4-difluorobenzyl bromide, compound 6b was synthesized as a white solid.

### Example 19

### Preparation of Compound 7a

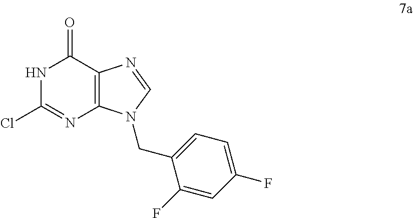

A 2-L three-necked round-bottomed flask equipped with a mechanical stirrer was charged with 2,6-dichloro-9-(2,4-difluorobenzyl)-9H-purine (6a) (31.0 g, 0.098 mol) and 0.2 N NaOH solution (984 mL). The reaction mixture was heated under reflux for 16 hours, filtered while hot through a sintered glass funnel and the filtrate allowed to cool to room temperature. The filtrate was acidified with glacial acetic acid (100 mL) and stirred at 0° C. for 5 hours. The precipitate was collected, washed with cold water (3×200 mL) and air-dried for 1 hour. Further drying in a vacuum oven at 55° C. for 16 hours provided 7a (25.1 g, 86%) as a white solid.

### Example 20

### Preparation of Compound 7b

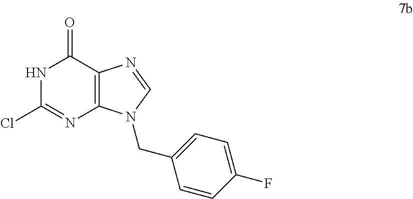

Using the method described in Example 19 and substituting compound 6b for compound 6a, compound 7b was synthesized as an off-white solid.

### Example 21

### Preparation of Compound 7c

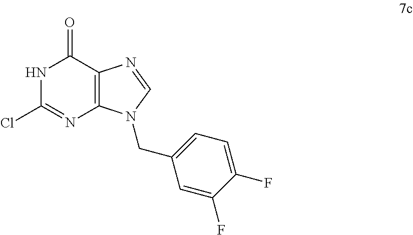

Using the method described in Example 19 and substituting 6c for compound 6a, compound 7c was synthesized as an off-white solid.

### Example 22

### Preparation of Compound 8a

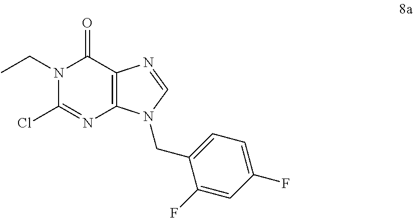

A 2-L three-necked round-bottomed flask equipped with a mechanical stirrer was charged with 2-chloro-9-(2,4-difluorobenzyl)-1H-purin-6(9H)-one (7a) (25.0 g, 0.084 mol), potassium carbonate (583 g, 0.421 mol) and N,N-dimethylacetamide (700 mL). The suspension was stirred under an inert atmosphere and iodoethane (15.8 g, 0.101 mol) was added. After stirring 16 hours at room temperature, the mixture was diluted with water (1 L) and then extracted with EtOAc (3×750 mL). The combined organic layers were washed with brine (3×750 mL), dried over Na₂SO₄ and filtered. The resulting brown solid was purified using column chromatography (silica gel, 10:90, hexanes, EtOAc) to provide compound 8a (13.4 g, 49%) as an off-white solid.

### Example 23

### Preparation of Compound 8b

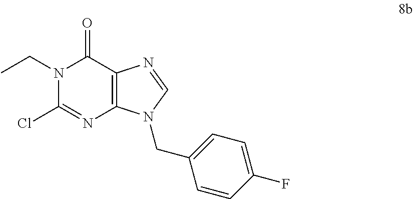

Using the method described in Example 22 and substituting compound 7b for compound 7a, compound 8b was synthesized as an off-white solid.

### Example 24

### Preparation of Compound 8c

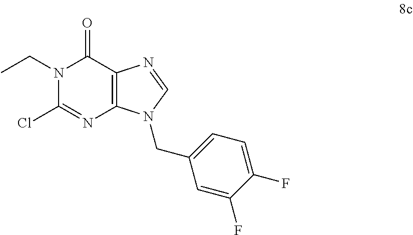

Using the method described in Example 22 and substituting compound 7c for compound 7a, compound 8c was synthesized as an off-white solid.

### Example 25

### Preparation of Compound 9a

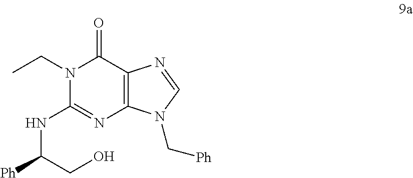

To a stirred solution of 9-benzyl-2-chloro-1-ethyl-1H-purin-6(9H)-one (4a) (925 mg, 3.20 mmol) in NMP (35 mL) under nitrogen atmosphere was added (R)-2-amino-2-phenylethanol (659 mg, 4.80 mmol) and DIPEA (1.1 mL, 6.40 mmol). The reaction mixture was place in an oil bath at 130° C. overnight, cooled to room temperature and poured into ice/water (500 mL). The mixture was stirred for 3 hours and the precipitate was collected and air-dried. The filter cake was dissolved in CH₂Cl₂ (200 mL), washed with brine (250 mL) and concentrated in vacuo to provide compound 9a (1.13 g, 90%) as an off-white solid.

### Example 26

### Preparation of Compound 9b

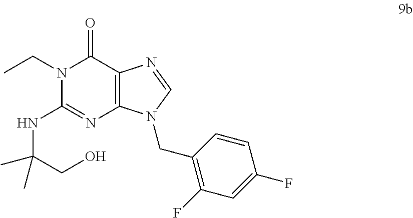

Using the method described in Example 25 and substituting compound 4e for compound 4a, and 2-amino-2,2,-dimethylethanol for (R)-2-amino-2-phenylethanol, compound 9b was synthesized as a light brown solid.

### Example 27

### Preparation of Compound 9c

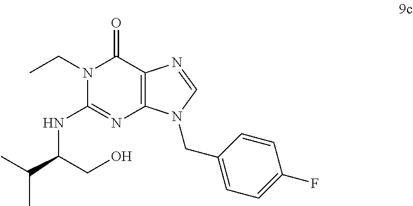

Using the method described in Example 25 and substituting compound 4b for compound 4a, and (R)-2-amino-2-isopropylethanol for (R)-2-amino-2-phenylethanol, compound 9c was synthesized as an off-white solid.

### Example 28

### Preparation of Compound 9d

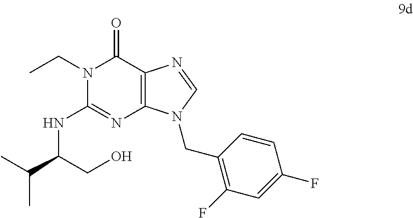

Using the method described in Example 25 and substituting compound 4c for compound 4a, and (R)-2-amino-2-isopropylethanol for (R)-2-amino-2-phenylethanol, compound 9d was synthesized as an off-white solid.

### Example 29

### Preparation of Compound 9e

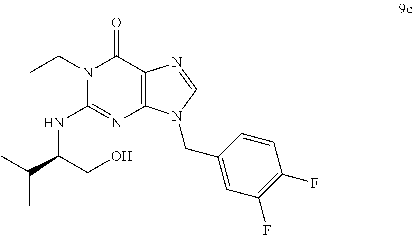

Using the method described in Example 28 and substituting compound 4d for compound 4a, compound 9e was synthesized as an off-white solid.

### Example 30

### Preparation of Compound 9f

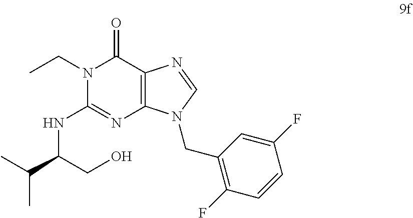

Using the method described in Example 28 and substituting compound 4e for compound 4a, compound 91 was synthesized as an off-white solid.

### Example 31

### Preparation of Compound 10a

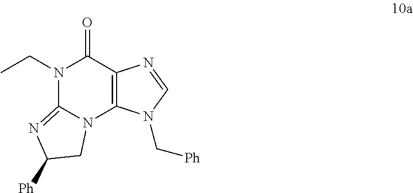

To a stirred solution of (R)-9-benzyl-1-ethyl-2-(2-hydroxy-1-phenylethylamino)-1H-purin-6(9H-one (9a) (1.13 g, 2.89 mmol) in CH₂Cl₂ (75 mL) at −10° C. was added thionyl chloride (1.04 g, 8.69 mmol) dropwise. The reaction mixture was stirred at −10° C. for 10 minutes, then allowed to warm to room temperature for 2 hours and concentrated in vacuo. The residue obtained was partitioned between sat. NaHCO₃ (100 mL) and CH₂Cl₂ (100 mL) and the layers separated. The aqueous phase was extracted with CH₂Cl₂ (100 mL) and the combined organic extracts were dried over Na₂SO₄, filtered and concentrated in vacuo to provide compound 10a (0.869 g, 81%) as an off-white solid.

### Example 32

### Preparation of Compound 10b

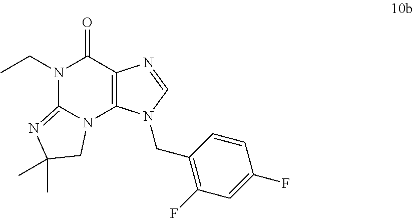

Using the method described in Example 31 and substituting compound 9b for compound 9a, compound 10b was synthesized as an off-white solid.

### Example 33

### Preparation of Compound 10e

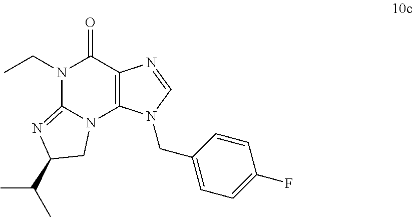

Using the method described in Example 25 and substituting compound 9c for compound 9a, compound 10c was synthesized as an off-white solid.

### Example 34

### Preparation of Compound 10d

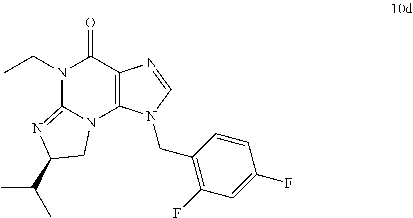

Using the method described in Example 25 and substituting compound 9d for compound 9a, compound 10d was synthesized as an off-white solid.

### Example 35

### Preparation of Compound 10e

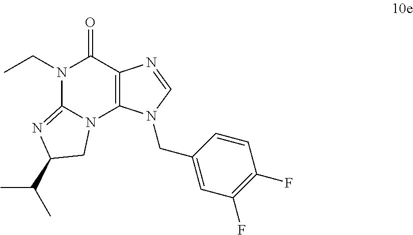

Using the method described in Example 25 and substituting compound 9e for compound 9a, compound 10e was synthesized as an off-white solid.

### Example 36

### Preparation of Compound 101

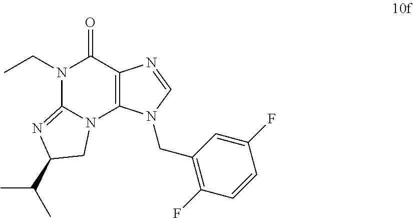

Using the method described in Example 25 and substituting compound 9f for compound 9a, compound 10f was synthesized as an off-white solid.

### Example 37

### Preparation of Compound 11a

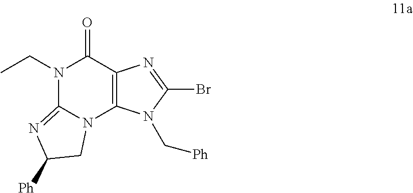

To a stirred solution of (R)-1-benzyl-5-ethyl-7-phenyl-7,8-dihydro-1H-imidazo[2,1-b]purin-4(5H)-one (10a) (0.869 g, 2.34 mmol) in CH₂Cl₂ (25 mL) at −10° C. was added N-bromosuccinimide (0.416 g, 2.34 mmol). The reaction mixture was stirred at −10° C. for 2 hours, warmed to room temperature and concentrated in vacuo. The residue obtained was purified using CombiFlash chromatography (silica gel, 80:10, ether/isopropanol) to provide compound 11a (1.10 g, >99%) as an off-white solid.

### Example 38

### Preparation of Compound 11b

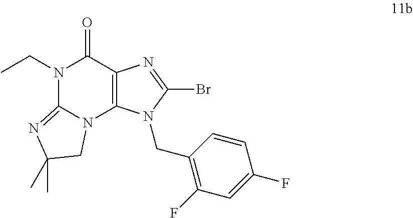

Using the method described in Example 37 and substituting compound 10b for compound 10a, compound 11b was synthesized as an off-white solid.

### Example 39

### Preparation of Compound 11c

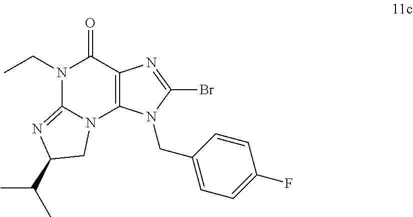

Using the method described in Example 37 and substituting compound 10c for compound 10a, compound 11c was synthesized as an off-white solid.

### Example 40

### Preparation of Compound 11d

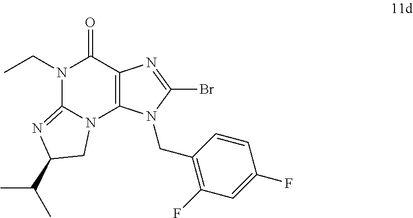

Using the method described in Example 37 and substituting compound 10d for compound 10a, compound 11e was synthesized as an off-white solid.

### Example 41

### Preparation of Compound 11e

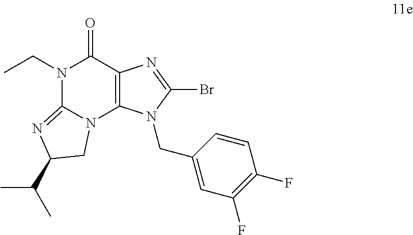

Using the method described in Example 37 and substituting compound 10e for compound 10a, compound 11e was synthesized as an off-white solid.

### Example 42

### Preparation of Compound 11f

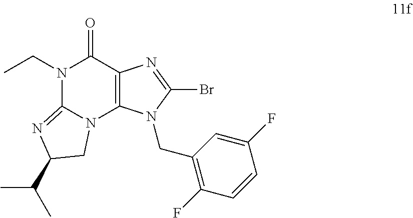

Using the method described in Example 37 and substituting compound 10f for compound 10a, compound 11f was synthesized as an off-white solid.

### Example 43

### Preparation of Compound 43a

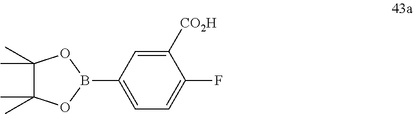

A 100 mL round-bottomed flask, equipped with a Dean-Stark apparatus, was charged with 5-borono-2-fluorobenzoic acid (1.00 g, 5.43 mmol), anhydrous toluene (50 mL) and pinacol (0.706 g, 5.98 mmol). The mixture was heated under reflux for 16 hours, cooled to room temperature, then concentrated in vacuo. The residue obtained was triturated with hexanes (200 mL) to provide compound 43a (1.33 g, 92%) as a white solid.

### Example 44

### Preparation of Compound 14a

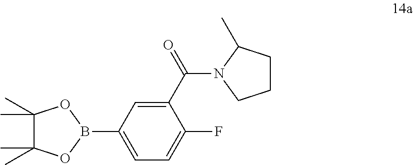

To a stirred solution of 2-fluoro-5-(4,4,5,5-tetramethyl-1,3,2-dioxaborolan-2-yl)benzoic acid (43a) (133 mg, 0.500 mmol) in anhydrous DMF (3 mL) was added HOBt (84 mg, 0.625 mmol), EDC∩HCl (120 mg, 0.625 mmol) and DIPEA (162 mg, 1.25 mmol). The reaction mixture was stirred for 10 minutes then 2-methylpyrrolidine (36 mg, 0.416 mmol) was added and the reaction was allowed to stir for an additional 16 hours. The reaction mixture was diluted with EtOAc (30 mL) and sat. NH₄Cl (20 mL) and the layers separated. The aqueous phase was extracted with EtOAc (20 mL) and the combined organic extracts were washed with brine (3×20 mL), dried over Na₂SO₄, filtered and concentrated in vacuo to provide compound 14a (146 mg, 87%) as a pale yellow oil.

### Example 45

### Preparation of Compound 14b

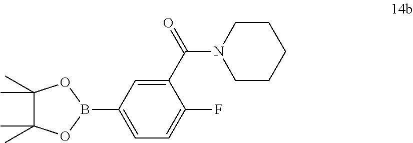

Using the method described in Example 44 and substituting piperidine for 2-methylpyrrolidine, compound 14b was synthesized as a light brown yellow oil.

### Example 46

### Preparation of Compound 14c

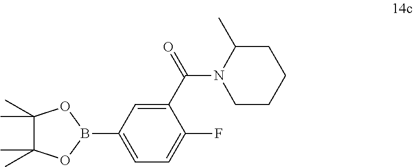

Using the method described in Example 44 and substituting 2-methylpiperidine for 2-methylpyrrolidine, compound 14e was synthesized as a pale yellow oil.

### Example 47

### Preparation of Compound 47a

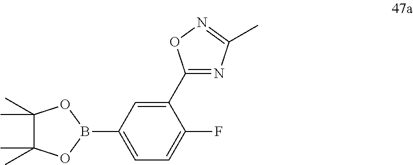

A 10 mL sealed tube was charged with 2-fluoro-5-(4,4,5,5-tetramethyl-1,3,2-dioxaborolan-2-yl)benzoic acid (43a) (200 mg, 0.752 mmol), HOBt (122 mg, 0.902 mmol), EDC•HCl (166 mg, 0.864 mmol), and CH₃CN (5 mL). The mixture was stirred at room temperature for 30 minutes and methylamidoxirne (56 mg, 902 mmol, see Organic Process Research & Development 2006, 10, 36-45) and powdered 4 Å molecular sieves was added. The sealed tube was placed in an oil bath at 85° C. for 2 days, then cooled to room temperature and poured into saturated NaHCO₃ (200 mL). The aqueous solution was extracted with EtOAc (2×125 mL) and the combined organic extracts were dried over Na₂SO₄, filtered and concentrated in vacuo. The crude product was purified using CombiFlash chromatography (silica gel, 50:50, CH₂Cl₂/EtOAc) to provide compound 47a (86 mg, 38%) as an off-white solid.

### Example 48

### Preparation of Compound 45

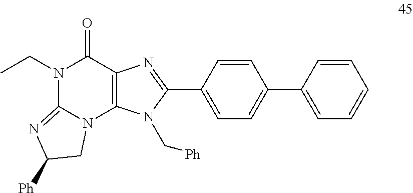

A 10 mL sealed tube was charged with (R)-1-benzyl-2-bromo-5-ethyl-7-phenyl-7,8-dihydro-1H-imidazo[2,1-b]purin-4(5H)-one (11a) (100 mg, 0.222 mmol), biphenyl-4-ylboronic acid (65 mg, 0.333 mmol), Pd(PPh₃)₄ (25 mg, 0.022 mmol), Na₂CO₃ (35 mg, 0.333 mmol), argon-degassed dimethoxyethane (2 mL) and argon-degassed water (0.5 mL). The tube was flushed with argon, sealed, and placed in an oil bath at 100° C. and allowed to remain at this temperature for about 15 hours. The reaction mixture was then cooled to room temperature, diluted with brine (5 mL) and CH₂Cl₂ (5 mL) and the layers separated. The aqueous phase was extracted with CH₂Cl₂ (5 mL) and the combined organic extracts were dried over Na₂SO₄, filtered and concentrated in vacuo. The residue obtained was purified using CombiFlash chromatography (silica gel, 95:5, CH₂Cl₂/MeOH) to provide an oil which was further purified using semi-preparative HPLC (Luna C18, CH₃CN/water with 0.05% TFA) to provide a solid. This solid was dissolved in a mixture of CH₃CN and water and the resulting solution freeze-dried overnight to provide compound 45 (64 mg, 55%) as a white solid: ¹H NMR (300 MHz, DMSO-d₆) δ 7.80-7.76 (m, 4H), 7.66-7.52 (m, 4H), 7.51-7.43 (m, 2H), 7.42-7.18 (m, 7H), 7.10 (d, J=7.1 Hz, 2H), 5.59-5.39 (m, 2H), 5.09 (t, J=7.7 Hz, 1H), 4.37 (t, J=9,3 Hz, 1H), 4.10-3.87 (m, 2H), 3.81 (t, J=8.2 Hz, 1H), 1.20 (t, J=6.9, 3H); APCI MS m/z 524 [M+H].

### Example 49

### Preparation of Compound 43

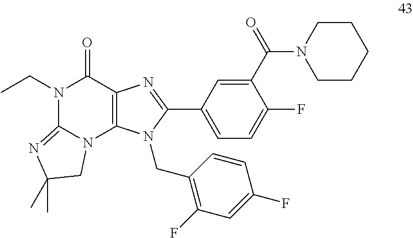

Using the method described in Example 48 and substituting compound 11b for compound 11a, and compound 14b for biphenyl-4-ylboronic acid, compound 43 was synthesized as an off-white solid: ¹H NMR (300 MHz, CDCl₃) [symbol] 7.54 (dd, J=6.2, 2.3 Hz, 1H), 7.47-7.40 (m, 1H), 7.12 (t, J=8.7 Hz, 1H), 6.97-6.86 (m, 2H), 6.78-6.68 (m, 1H), 5.28 (s, 2H), 4.08 (q, J=7.0 Hz, 2H), 3.75-3.66 (m, 2H), 3.57 (s, 2H), 3.27-3.20 (m, 2H), 1.70-1.54 (m, 6H), 1.31-1.25 (m, 9H); ESI MS m/z 565 [M+H]⁺.

### Example 50

### Preparation of Compound 46

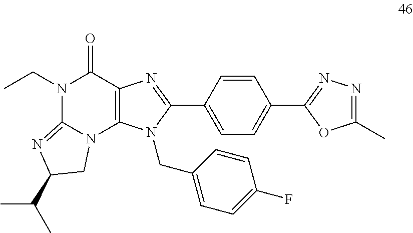

Using the method described in Example 48 and substituting compound 11c for compound 11a, and compound 47a for biphenyl-4-ylboronic acid, a crude compound was synthesized as an off-white solid, then dissolved in CH₃CN and water and the resulting solution freeze-dried overnight to provide compound 46 (59 mg, 50%) as a white solid: ¹H NMR (300 MHz, CDCl₃) δ 8.04 (d, J=8.6 Hz, 2H), 7.67 (d, J=8.6 Hz, 2H), 7.20-7.12 (m, 2H), 7.11-7.02 (m, 2H), 5.39 (d, J=17.6 Hz, 2H), 4.27-4.13 (m, 1H), 4.09-3.90 (m, 2H), 3.90-3.79 (m, 1H), 3.52 (dd, J=8.0, 6.2 Hz, 1H), 2.62 (s, 3H), 1.80-1.66 (m, 1H), 1.30 (t, J=7.0 Hz, 3H), 0.81 (d, J=6.8 Hz, 3H), 0.72 (d, J=6.8 Hz, 3H); ESI MS m/z 514 [M+H]⁺.

### Example 51

### Preparation of Compound 16

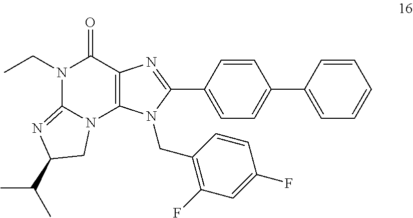

Using the method described in Example 48 and substituting compound 11d for compound 11a, compound 16 was synthesized as an off-white solid: ¹H NMR (300 MHz, CDCl₃) i 7.65-7.57 (m, 6H), 7.47-7.42 (m, 3H), 6.95-6.85 (m, 3H), 5.40 (s, 2H), 4.24-4.17 (m, 1H), 4.04-3.93 (m, 2H), 3.89-3.83 (m, 1H), 3.54 (dd, J=7.8, 6.2 Hz, 1H), 1.79-1.72 (m, 1H), 1.30 (t, J=7.0 Hz, 3H), 0.84 (d, J=6.8 Hz, 3H), 0.74 (d, J=6.8 Hz, 3H); APCI MS m/z 526 [M+H]⁺.

### Example 52

### Preparation of Compound 47

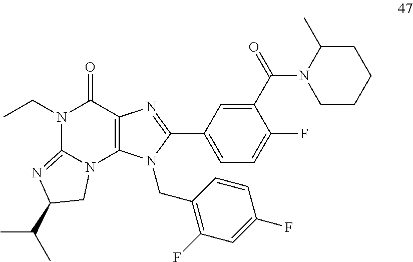

Using the method described in Example 48 and substituting substituting compound 11d for compound 11a, and compound 14c for biphenyl-4-ylboronic acid, a residue was obtained as an off-white solid. The residue obtained was purified using CombiFlash chromatography (silica gel, 90:10 CH₂Cl₂/MeOH) to provide a crude oil which was further purified using semi-preparative HPLC (Luna C18, CH₃CN/water with 0.05% TFA) to provide an oil. This oil was dissolved in CH₃CN and water and the resulting solution freeze-dried overnight to provide compound 47 (74 mg, 35%) as a white solid: ¹H NMR (300 MHz, CDCl₃) δ 7.60-7.47 (m, 2H), 7.17-7.07 (m, 1H), 6.96-6.87 (m, 2H), 6.71 (m, 1H), 5.31 (s, 2H), 5.01-4.54 (m, 1H), 4.24-4.13 (m, 1H), 4.03-3.77 (m, 4H), 3.54 (m, 1H), 3.24-2.76 (m, 2H), 1.78-1.60 (m, 6H), 1.31-1.24 (m, 6H), 0.94 (d, J=6.6 Hz, 3H), 0.74 (d, J=6.6 Hz, 3H); ESI MS m/z 593 [M+H]⁺.

### Example 53

### Preparation of Compound 48

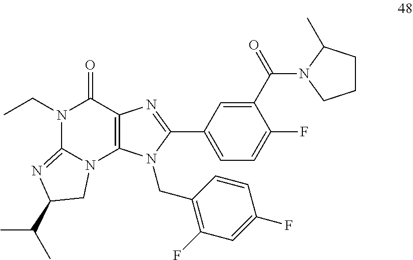

Using the method described in Example 48 and substituting compound 11d for compound 11a, and compound 14a for biphenyl-4-ylboronic acid a residue was obtained as an off-white solid. The residue obtained was purified using CombiFlash chromatography (silica gel, 90:10 CH₂Cl₂/MeOH) to provide a crude oil which was further purified using semi-preparative HPLC (Luna C18, CH₃CN/water with 0.05% TFA) to provide a colorless oil. This oil was dissolved in CH₃CN and water and the resulting solution freeze-dried overnight to provide compound 48 (67 mg, 31%) as an off-white solid: ¹H NMR (300 MHz, CDCl₃) δ 7.63-7.41 (m, 2H), 7.15-7.11 (m, 1H), 6.95-6.89 (m, 2H), 6.88-6.72 (m, 1H), 5.32 (s, 2H), 4.32-4.15 (m, 2H), 4.04-3.83 (m, 3H), 3.70-3.35 (m, 3H), 2.13-1.72 (m, 4H), 1.33-1.26 (m, 6H), 0.91-0.73 (M, 7H); ESI MS m/z 579 [M+H]⁺.

### Example 54

### Preparation of Compound 49

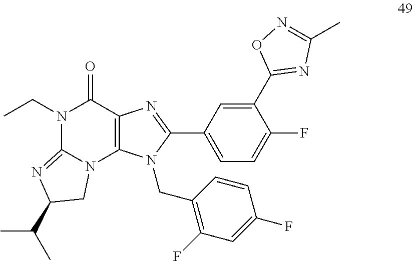

Using the method described in Example 48 and substituting substituting compound 11d for compound 11a, and compound 47a for biphenyl-4-ylboronic acid, a residue was obtained as an off-white solid. The residue obtained was purified using CombiFlash chromatography (silica gel, 97:3 CH₂Cl₂/MeOH) to provide a crude oil which was further purified using semi-preparative HPLC (Luna C18, CH₃CN/water with 0.05% TFA) to provide a solid. This solid was dissolved in CH₃CN and water and the resulting solution freeze-dried overnight to provide compound 49 (40 mg, 33%) as an off-white solid: ¹H NMR (300 MHz, CDCl₃) δ 8.20 (dd, J=6.5, 2.3 Hz, 1H), 7.85-7.77 (m, 1H), 7.33 (dd, J=9.8, 8.9 Hz, 1H), 7.01-6.89 (m, 2H), 6.85-6.74 (m, 1H), 5.38 (s, 2H), 4.29-4.13 (m, 1H), 4.07-3.86 (m, 3H), 3.60 (dd, J=7.6, 6.4 Hz, 1H), 2.47 (s, 3H), 1.85-1.71 (m, 1H), 1.31 (t, J=7.0 Hz, 3H), 0.87 (d, J=6.8 Hz, 3H), 0.78 (d, J=6.8 Hz, 3H); ESI MS m/z 550 [M+H]⁺.

### Example 55

### Preparation of Compound 50

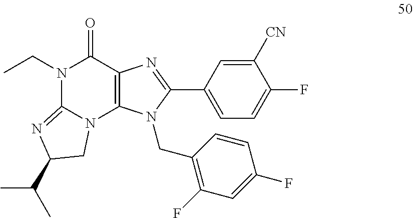

Using the method described in Example 48 and substituting substituting compound 11d for compound 11a, and 3-cyano-4-fluorophenylboronic acid for biphenyl-4-ylboronic acid, a residue was obtained as an off-white solid. The residue obtained was purified using CombiFlash chromatography (silica gel, 1:3 CH₂Cl₂/EtOAc) to provide compound 50 (280 mg, 80%) as an off-white solid.

### Example 56

### Preparation of Compound 51

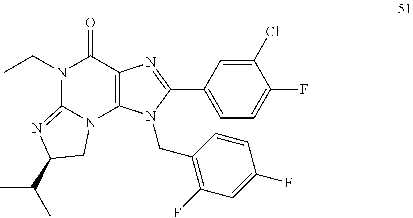

Using the method described in Example 48 and substituting substituting compound 11d for compound 11a, and 3-chloro-4-fluorophenylboronic acid for biphenyl-4-ylboronic acid, a residue was obtained as yellow solid. The residue obtained was purified using CombiFlash chromatography (silica gel, 95:5 CH₂Cl₂/MeOH) to provide compound 51 (1.50 g, 79%) as a light yellow solid.

### Example 57

### Preparation of Compound 52

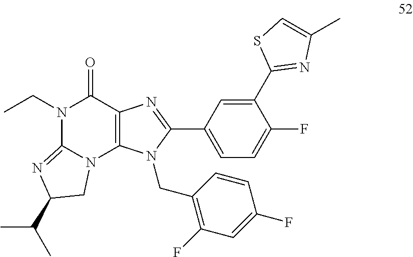

A 10 mL microwave vial was charged with (R)-5-(1-(2,4-difluorobenzyl)-5-ethyl-7-isopropyl-4-oxo-4,5,7,8-tetrahydro-1H-imidazo[2,1-b]purin-2-yl)-2-fluorobenzonitrile (50) (275 mg, 0.558 mmol), MeOH (5 mL), and ammonium sulfide solution in water (20 wt %, 600 μL, 1.67 mmol). The reaction mixture was stirred in a microwave at 85° C. for 15 minutes, cooled to room temperature and concentrated in vacuo. The residue obtained was diluted with EtOAc (200 mL) and brine (250 mL) and the layers separated. The aqueous phase was extracted with EtOAc (200 mL) and the combined organic extracts were dried over Na₂SO₄, filtered and concentrated in vacuo. The crude product was purified using CombiFlash chromatography (silica gel, 96:4, CH₂Cl₂/MeOH) to provide (R)-5-(1-(2,4-difluorobenzyl)-5-ethyl-7-isopropyl-4-oxo-4,5,7,8-tetrahydro-1H-imidazo[2,1-b]purin-2-yl)-2-fluorobenzothioamide (118 mg, 40%) as a yellow solid.

A 10 mL sealed tube was charged with the above thioamide (115 mg, 0.218 mmol), CH₃CN (5 mL) and chloroacetone (19 μL, 0.240 mmol). The sealed tube was placed in an oil bath at 85° C. for 16 hours, then cooled to room temperature and diluted with CH₂Cl₂ (100 mL) and brine (200 mL). After separating the layers, the aqueous phase was extracted with CH₂Cl₂ (100 mL) and the combined organic extracts were dried over Na₂SO₄, filtered and concentrated in vacuo. The crude product was purified using CombiFlash chromatography (silica gel, 96:4, CH₂Cl₂/MeOH) and semi-preparative HPLC (Luna C18, CH₃CN/water with 0.05% TFA) to provide a solid. This solid was dissolved in a mixture of CH₃CN and water and the resulting solution freeze-dried overnight to provide compound 52 (46 mg, 37%) as a white solid: ¹H NMR (300 MHz, CDCl₃) δ 8.38 (dd, J=6.9, 2.4 Hz, 1H), 7.71-7.63 (m, 1H), 7.30-7.19 (m, 1H), 7.02-6.89 (m, 3H), 6.88-6.77 (m, 1H), 5.40 (s, 2H), 4.28-4.14 (m, 1H), 4.09-3.94 (m, 2H), 3.95-3.85 (m, 1H), 3.59 (dd, J=7.7, 6.3 Hz, 1H), 2.43 (s, 3H), 1.85-1.71 (m, 1H), 1.31 (t, J=7.0 Hz, 3H), 0.86 (d, J=6.8 Hz, 3H), 0.77 (d, J=6.8 Hz, 3H); ESI MS m/z 565 [M+H]⁺.

### Example 58

### Preparation of Compound 53

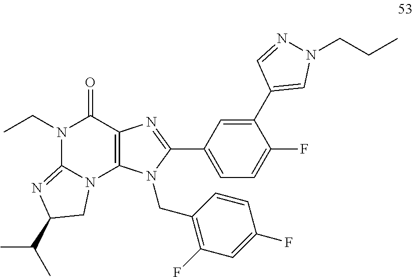

A 10 mL sealed tube was charged with (R)-2-(3-chloro-4-fluorophenyl)-1-(2,4-difluorobenzyl)-5-ethyl-7-isopropyl-7,8-dihydro-1H-imidazo[2,1-b]purin-4(5H)-one (5H) (150 mg, 0.299 mmol), 1-propyl-4-(4,4,5,5-tetramethyl)-1,3,2-dioxaborolan-2-yl (106 mg, 0.448 mmol), K₃PO₄ (127 mg, 0.598 mmol), Pd₂(dba)₃ (2.74 mg, 2.99 μmol), X-Phos (5.70 mg, 12.0 μmol), and argon-degassed n-butanol (1.2 mL). The tube was placed in an oil bath at 100° C. for 16 hours, cooled to room temperature and diluted with CH₂Cl₂ (250 mL). The organic mixture was washed with brine (2×100 mL), dried over Na₂SO₄, filtered and concentrated in vacuo. The residue obtained was purified using CombiFlash chromatography (silica gel, 50:50, hexanes/EtOAc) to provide a crude oil. This oil was dissolved in a mixture of CH₃CN and water and the resulting solution freeze-dried overnight to provide compound 53 (52 mg, 30%) as a white solid: ¹H NMR (300 MHz, DMSO-d_{.6}) δ 8.11 (d, J=1.8 Hz, 1H), 7.78-7.68 (m, 2H), 7.40-7.22 (m, 3H), 7.12-6.96 (m, 2H), 5.46 (s, 2H), 4.17-3.80 (m, 6H), 3.77-3.64 (m, 1H), 1.89-1.73 (m, 2H), 1.72-1.58 (m, 1H), 1.17 (t, J=6.9 Hz, 3H), 0.88-0.79 (m, 6H), 0.75 (d, J=6.6 Hz, 3H); ESI MS m/z 576 [M+H]⁺.

### Example 59

### Preparation of Compound 54

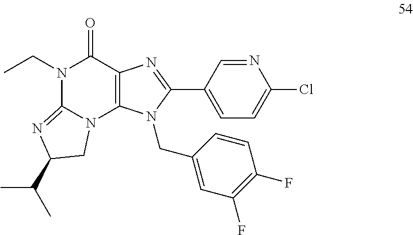

The title compound was prepared from (R)-2-bromo-1-(3,4-difluorobenzyl)-5-ethyl-7-isopropyl-7,8-dihydro-1H-imidazo[2,1-b]purin-4(5H)-one (11e) (500 mg, 1.11 mmol) and 2-chloropyridine-5-boronic acid (174 mg, 1.11 mmol) heating for 6 hours according to the procedure described in Example 48. The residue obtained was purified using CombiFlash chromatography (silica gel, 95:5, CH₂Cl₂/MeOH) and semi-preparative HPLC (Luna C18, CH₃CN/water with 0.05% TFA) to provide a solid. This solid was dissolved in a mixture of CH₃CN and water and the resulting solution freeze-dried overnight to provide compound 54 (160 mg, 30%) as a white solid: ¹H NMR (300 MHz, CDCl₃) δ 8.43 (d, J=2.0 Hz, 1H), 7.91 (dd, J=8.3, 2.5 Hz, 1H), 7.39 (d, J=8.3 Hz, 1H), 7.30-7.20 (m, 1H), 6.97-6.87 (m, 1H), 6.83-6.74 (m, 1H), 5.41-5.29 (m, 2H), 4.27-4.13 (m, 1H), 4.07-3.92 (m, 2H), 3.84 (dd, J=9.6, 8.1 Hz, 1H), 3.53 (dd, J=8.0, 6.6 Hz, 1H), 1.84-1.69 (m, 1H), 1.30 (t, J=7.0 Hz, 3H), 0.85 (d, J=6.8 Hz, 3H), 0.77 (d, J=6.8 Hz, 3H); ESI MS m/z 485 [M+H]⁺.

### Example 60

### Preparation of Compound 55

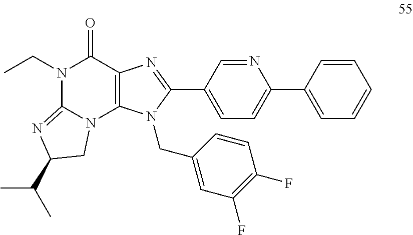

The title compound was prepared from (R)-2-(6-chloropyridin-3-yl)-1-(3,4-difluorobenzyl)-5-ethyl-7-isopropyl-7,8-dihydro-1H-imidazo[2,1-b]purin-4(5H)-one (54) (65 mg, 0.134 mmol) and phenylboronic acid (20 mg, 0.161 mmol) heating for 4 hours according to the procedure described in Example 48. The residue obtained was purified using CombiFlash chromatography (silica gel, 96:4, CH₂Cl₂/MeOH) to provide a solid. The solid was dissolved in a mixture of CH₃CN and water and the resulting solution freeze-dried overnight to provide compound 23 (61 mg, 86%) as a yellow solid: ¹H NMR (300 MHz, CDCl₃) δ 8.72 (d, J=1.8 Hz, 1H), 8.07-7.97 (m, 3H), 7.81 (d, J=8.3 Hz, 1H), 7.53-7.42 (m, 3H), 7.33-7.21 (m, 1H), 7.01-6.91 (m, 1H), 6.87-6.79 (m, 1H), 5.39 (d, J=18.0 Hz, 2H), 4.29-4.15 (m, 1H), 4.10-3.92 (m, 2H), 3.85 (t, J=8.8 Hz, 1H), 3.59-3.49 (m, 1H), 1.82-1.69 (m, 1H), 1.31 (t, J=7.0 Hz, 3H), 0.85 (d, J=6.8 Hz, 3H), 0.76 (d, J=6.8 Hz, 3H); ESI MS m/z 527 [M+H]⁺.

### Example 61

### Preparation of Compound 18

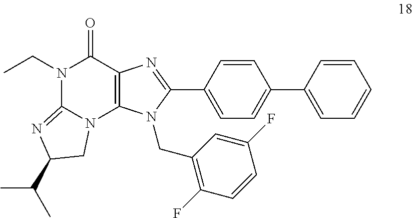

The title compound was prepared from (R)-2-bromo-1-(2,5-difluorobenzyl)-5-ethyl-7-isopropyl-7,8-dihydro-1H-imidazo[2,1-b]purin-4(5H)-one (11f) (91 mg, 0.200 mmol) and biphenyl-4-ylboronic acid (48 mg, 0.240 mmol) heating for 3 hours according to the procedure described in Example 48. The residue obtained was purified using CombiFlash chromatography (silica gel, 95:5 CH₂Cl₂/MeOH) to provide a solid. The solid was dissolved in a mixture of CH₃CN and water and the resulting solution freeze-dried overnight to provide compound 18 (57 mg, 54%) as a white solid: ¹H NMR (300 MHz, CDCl₃) δ 7.63-7.58 (m, 6H), 7.44-7.36 (m, 3H), 7.08 (m, 2H), 6.58 (m, 1H), 5.42 (s, 2H), 4.24-4.05 (m, 1H), 4.03-3.93 (m, 2H), 3.89-3.83 (m, 1H), 3.54 (dd, J=7.8, 6.3 Hz, 1H), 1.79-1.72 (m, 1H), 1.30 (t, J=7.0 Hz, 3H), 0.84 (d, J=6.7 Hz, 3H), 0.74 (d, J=6.7 Hz, 3H); ESI MS m/z 526 [M+H]⁺.

### Example 62

### Preparation of Compound 62A

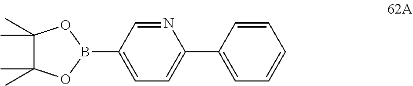

### Step A—Preparation of 5-Chloro-2-phenylpyridine

A 100-mL, round-bottomed flask, fitted with a reflux condenser was charged with 2,5-dichloropyridine (1.00 g, 6.76 mmol), phenylboronic acid (1.07 g, 8.78 mmol), [1,4-bis(diphenylphosphino)butane]palladium(II) dichloride (0.204 g, 0.338 mmol), argon-degassed toluene/ethanol (4:1, 17 mL) and argon-degassed aqueous 1 N sodium carbonate (6.6 mL). The mixture was heated to reflux for 3 h, cooled to room temperature and diluted with water (15 mL) and EtOAc (100 mL). The layers separated and the aqueous phase was extracted with EtOAc (100 mL). The combine organic extracts were dried over Na₂SO₄, filtered and concentrated. Purification by flash column chromatography (silica gel, 50:50 hexanes/CH₂Cl₂) provided 5-chloro-2-phenylpyridine (0.959 g, 75%) as a white solid.

### Step B—Preparation of Compound 62A

A 150 mL sealed tube was charged with bis(dibenzylideneacetone)palladium (87 mg, 0.152 mmol), tricyclohexylphosphine (102 mg, 0.364 mmol) and nitrogen-degassed 1,4-dioxane (32 mL). After stirring at room temperature for 30 min under nitrogen, bis(pinacolato)diboron (1.41 g, 5.56 mmol), KOAc (0.744 g, 7.59 mmol) and 5-chloro-2-phenylpyridine (0.959 g, 5.06 mmol, from Step A) was added to the reaction mixture. The tube was sealed and place in an oil bath at 80° C. for 16 hours. The mixture was cooled to room temperature, diluted with brine (50 mL) and CH₂Cl₂ (100 mL) and the layers were separated. The aqueous phase was extracted with CH₂Cl₂ (100 mL) and the combined organics washed with brine (150 mL), dried over Na₂SO₄, filtered and concentrated. Purification by flash column chromatography (silica gel, 50:50 CH₂Cl₂/EtOAc) provided compound 62A (0.569 g, 40%) as a white solid.

### Example 63

### Preparation of Compound 60

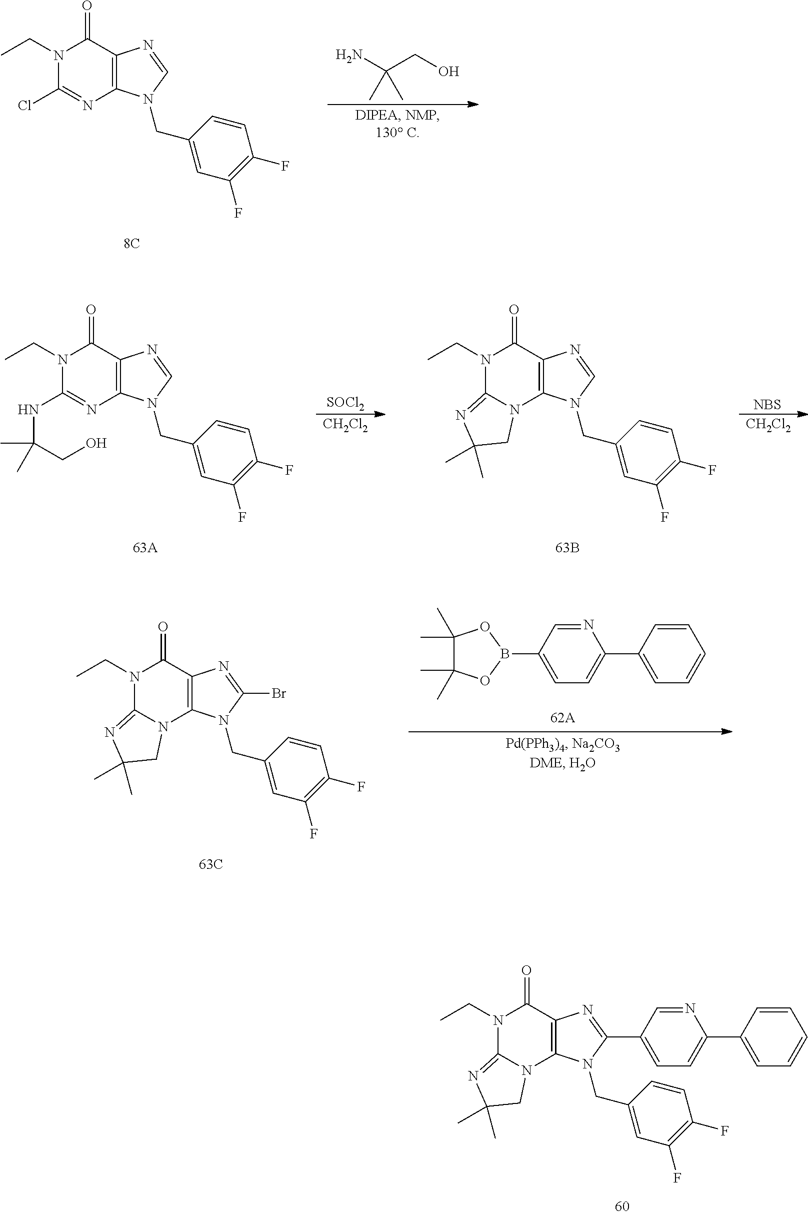

### Step A—Preparation of Compound 63A

To a stirred solution of compound 8C (6.70 g, 20.6 mmol) in NMP (125 mL) was added 2-amino-2-methylpropan-1-ol (4.59 g, 51.5 mmol) and DIPEA (8.00 g, 61.9 mmol). The resulting reaction was heated to 130° C. and allowed to stir at this temperature for 16 hours, then cooled to room temperature and poured into water (500 mL). The resulting solution was stirred for 3 hours and the precipitate formed was collected by filtration, washed with water (200 mL) and air-dried for 1 hour. Further drying in a vacuum oven at 50° C. provided compound 63A (6.62 g, 85%) as a white solid.

### Step B—Preparation of Compound 63B

To a stirred solution of compound 63A (6.62 g, 17.5 mmol) in CH₂Cl₂ (400 mL) was added thionyl chloride (6.26 g, 52.6 mmol). The resulting reaction was stirred at room temperature for 20 hours, then concentrated in vacuo and the residue obtained was partitioned between saturated NaHCO₃ (400 mL) and CH₂Cl₂ (400 mL). The layers were separated and the aqueous phase was extracted with CH₂Cl₂ (3×200 mL). The combined organic extracts were dried over Na₂SO₄, filtered and concentrated in vacuo to provide compound 6313 (6.85 g, >99%) as a white solid.

### Step C—Preparation of Compound 63C

To a stirred solution of compound 63B (6.85 g, 19.0 mmol) in CH₂Cl₂ (240 mL) was added NBS (3.39 g, 19.0 mmol). The resulting reaction was allowed to stir for 3 hours at room temperature, then was diluted with brine (200 mL) and the layers were separated. The organic phase was washed with brine (3×100 mL), dried over Na₂SO₄, filtered and concentrated in vacuo. The resulting residue was purified using flash column chromatography (silica gel, 95:5 CH₂Cl₂/MeOH) to provide compound 63C (7.15 g, 93%) as a white solid.

### Step D—Preparation of Compound 60

A 10 mL sealed tube was charged with compound 63C (110 mg, 0.251 mmol), compound 62A (85 mg, 0.301 mmol, from Example 62), Na₂CO₃ (53 mg, 0.502 mmol), Pd(PPh₃)₄ (29 mg, 0.025 mmol) and argon-degassed dimethoxyethane/water (2:1, 5 mL). The tube was placed in an oil bath at 100° C. and allowed to remain at this temperature for 16 hours, then the tube was removed from the heat bath and allowed to cool to room temperature. The cooled reaction mixture was diluted with brine (50 mL) and CH₂Cl₂ (50 mL), then separated and the aqueous phase was extracted with CH₂Cl₂ (2×50 mL). The combined organic extracts were dried over Na₂SO₄, filtered and concentrated in vacuo. The residue obtained was purified using flash column chromatography (silica gel, 20:80, CH₂Cl₂/EtOAc) to provide a crude oil which was further purified using semi-preparative HPLC (Luna C18, CH₃CN/water with 0.05% TFA) to provide a solid product. This solid product was dissolved in a mixture of CH₃CN and water and the resulting solution was freeze-dried for about 15 hours to provide compound 60 (49 mg, 38%) as an off-white solid: m.p. 124-128° C.; ¹H NMR (300 MHz, CDCl₃) δ 8.70 (s, 1H), 8.03-7.98 (m, 3H), 7.80 (d, J=8.4 Hz, 1H), 7.50-7.44 (m, 3H), 7.28 (m, 1H), 6.98-6.93 (m, 1H), 6.84-6.81 (m, 1H), 5.36 (s, 2H), 4.09 (q, J=6.9 Hz, 2H), 3.55 (s, 2H), 1.30 (t, J=6.9 Hz, 3H), 1.25 (s, 6H); EST MS m/z 513 [M+H]⁺.

### Example 64

### P2X₇ Inhibition Assay

The ability of the compounds of this invention to inhibit P2X₇ inhibitory can be determined using a P2X₇-HEK-293 stable cell line in Ca⁺⁺ flux assay that can be carried out as described in Cheewatrakoolpong et al., Biochem. Biophys. Res. Commun. 332:17-27 (2005).

Using this method, illustrative Compounds of Formula (I) were tested and measured IC₅₀ values ranged from about 10 nM to about 1 mM. Compounds 161-185 demonstrated IC₅₀ values ranging from about 10 nM to about 100 nM. By way of example the following compounds had IC₅₀ values of:

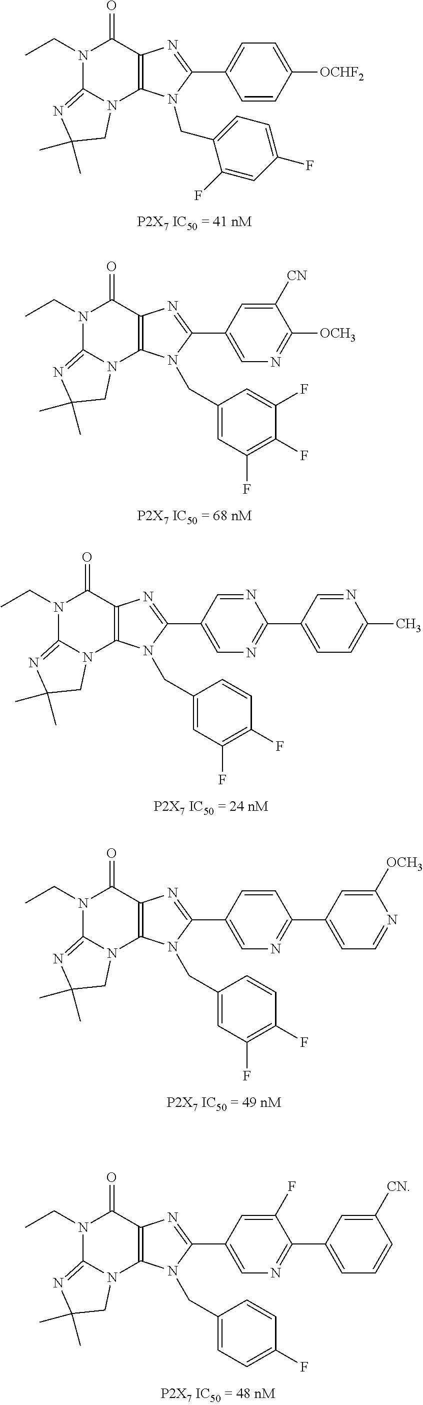

### Uses of the Compounds of Formula (I)

The Compounds of Formula (I) are useful in human and veterinary medicine for treating or preventing a Condition in a patient. In accordance with the invention, the Compounds of Formula (I) can be administered to a patient in need of treatment or prevention of a Condition.

### Treatment of Pain

The Compounds of Formula (I) are useful for treating or preventing pain in a patient. Accordingly, in one embodiment, the present invention provides a method for treating pain in a patient, comprising administering to the patient an effective amount of one or more Compounds of Formula (I).

In another embodiment, the present invention provides a method for treating pain in a patient, comprising administering to the patient an effective amount of one or more of illustrative compounds 1-160.

In another embodiment, the present invention provides a method for treating pain in a patient, comprising administering to the patient an effective amount of one or more of illustrative compounds 1.61-185.

Non-limiting examples of pain treatable or preventable using the present methods, include acute pain, back pain, chronic pain, fibromyalgia, post-herpatic neuralgia, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache, inflammatory pain, pain caused by diabetes, pain caused by arthritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

In still another embodiment, the pain is chronic pain.

In yet another embodiment, the pain is pain cause by arthritis.

In another embodiment, the pain is pain cause by diabetes.

In a further embodiment, the pain is inflammatory pain.

Neuropathic pain as used herein refers to an abnormal state of pain sensation, in which a reduction of pain threshold and the like are continued, due to functional abnormalities accompanying damage or degeneration of a nerve, plexus or perineural soft tissue, which is caused by wound (e.g., lacerations, contusions, nerve avulsion injuries, amputation of a limb), compression (carpal tunnel syndrome, trigeminal neuralgia, tumor activity), infection, cancer, ischemia and the like, or metabolic disorders such as diabetes mellitus and the like. Neuropathic pain includes pain caused by central nerve damage, peripheral nerve damage, diabetic neuropathy, mononeuropathy or polyneuropathy. In one embodiment, the neuropathic pain is induced by diabetes.

Other examples of neuropathic pain treatable or preventable using the Compounds of Formula (I) include, but are not limited to, pain caused by naturopathic therapy, pain that is resistant to naturopathic therapy, allodynia (a pain sensation induced by mechanical or thermal stimulus that does not normally provoke pain), hyperalgesia (an excessive response to a stimulus that is normally painful), hyperesthesia (an excessive response to a contact stimulus), diabetic polyneuropathy, entrapment neuropathy, central pain, labor pain, myocardial infarction pain, post-stroke pain, pancreatic pain, colic pain, muscle pain, post-operative pain, post-stroke pain, pain associated with Parkinson's disease, pain associated with intensive care, pain associated with a periodontal disease (including gingivitis and periodontitis), menstrual pain, migraine pain, persistent headaches (e.g., cluster headache or chronic tension headache), persistent pain states (e.g., fibromyalgia or myofascial pain), trigeminal neuralgia, postherpetic neuralgia, bursitis, pain associated with AIDS, pain associated with multiple sclerosis, pain due to spinal trauma and/or degeneration, burn pain, referred pain, enhanced memory of pain and neuronal mechanisms involved in coping with pain. Inflammatory pain may arise as a result of soft tissue injury including that involving the musculature (myositis) and viscera (colitis and inflammatory bowel disease, pancreatitis, cystitis, ileitis, Crohn's disease), nerves (neuritis, radiculopathies, radioculogangionitis), arthritic conditions (e.g. rheumatoid disease and related conditions such as ankylosing spondylitis), joint disease (including osteoarthritis). In specific embodiments, the Compounds of Formula (I) are useful for treating or preventing allodynia or hyperalgesia.

### Treatment of an Inflammatory Disease

The Compounds of Formula (I) can be useful for treating or preventing an inflammatory diesase in a patient. Accordingly, in one embodiment, the present invention provides a method for treating an inflammatory diesase in a patient, comprising administering to the patient an effective amount of one or more Compounds of Formula (I).

In another embodiment, the present invention provides a method for treating an inflammatory diesase in a patient, comprising administering to the patient an effective amount of one or more of illustrative compounds 1460.

In another embodiment, the present invention provides a method for treating an inflammatory diesase in a patient, comprising administering to the patient an effective amount of one or more of illustrative compounds 161-185.

Non-limiting examples of inflammatory diseases treatable or preventable using the present methods, include diabetic neuropathy; arthritis, such as osteoarthritis, rheumatoid arthritis, septic arthritis, gout, pseudogout, juvenile arthritis or Still's disease; inflammatory bowel diseases, such as ileitis, Crohn's disease and ulcerative colitis; organ transplant rejection; inflammatory bowel disease; inflammatory lung diseases such as asthma, adult respiratory distress syndrome and chronic obstructive pulmonary disease (COPD); inflammatory diseases of the eye, such as, corneal dystrophy, trachoma, uveitis and sympathetic ophthalmitis; chronic inflammatory diseases of the gum, such as gingivitis and periodontitis; inflammatory diseases of the kidney, such as glomerulonephritis and nephrosis; inflammatory diseases of the skin, such as sclerodermatitis, psoriasis and eczema; renal colic; reperfusion injury; pyrexia; ischemic injury; multiple sclerosis; systemic lupus erythematosis; periodic fever syndromes, such as chronic infantile neurological cutaneous and articular syndrome (CINCA), familial cold autoinflammatory syndrome (FCAS), Muckle-Wells Syndrome (MWS), familial Mediterranean fever (FMF) and pyrogenic arthritis, pyroderma gangrenosum and acne syndrome (PAPA); and inflammatory arthropathies, such as ankylosing spondylitis, psoriatric arthritis and Reiter's syndrome.

The term “inflammatory disease” as used herein included both local inflammatory responses and systemic inflammation.

In one embodiment, the inflammatory disease is arthritis.

In another embodiment, the inflammatory disease is rhematoid arthritis.

In another embodiment, the inflammatory disease is osteoarthritis.

In still another embodiment, the inflammatory disease is asthma.

In yet another embodiment, the inflammatory disease is chronic obstructive pulmonary disease (COPD).

In another embodiment, the inflammatory disease is inflammatory bowel disease.

### Combination Therapy

In one embodiment, the present invention provides methods for treating a Condition in a patient, the method comprising administering to the patient one or more Compounds of Formula (I), or a pharmaceutically acceptable salt, solvate, ester or prodrug thereof and at least one additional therapeutic agent that is not a Compound of Formula (I), wherein the amounts administered are together effective to treat or prevent a Condition.

In one embodiment, the at least one additional therapeutic agent comprises an analgesic agent.

Additional analgesic agents useful in the present methods for treating pain include, but are not limited to, non-opioid (also known as non-steroidal anti-inflammatory agents) analgesics such as acetylsalicylic acid, choline magnesium trisalicylate, acetaminophen, ibuprofen, fenoprofen, diflusinal, and naproxen; opioid analgesics such as morphine, hydromorphone, methadone, levorphanol, fentanyl, oxycodone, and oxymorphone; steroids such as prednisolone, fluticasone, triamcinolone, beclomethasone, mometasone, budisamide, betamethasone, dexamethasone, prednisone, flunisolide and cortisone; COX-I inhibitors such as aspirin and piroxicam; and COX-II inhibitors such as rofecoxib, celecoxib, valdecoxib and etoricoxib.

Other analgesic agents useful in the present methods for treating pain include, but are not limited to, gabapentin, pregabalin and duloxetine.

In one embodiment, the other analgesic agent is an opioid analgesic. In another embodiment, the other analgesic agent is a non-opioid analgesic. In another embodiment, the other analgesic agent is a COX-I inhibitor. In still another embodiment, the other analgesic agent is a COX-II inhibitor. In yet another embodiment, the other analgesic agent is selected from aspirin, acetaminophen, ibuprofen, fenoprofen, naproxen, morphine, hydromorphone, methadone, levorphanol, fentanyl, oxycodone, and oxymorphone.

In one embodiment, the at least one additional therapeutic agent comprises an anti-inflammatory agent.

Non-limiting examples of additional anti-inflammatory agents useful in the present methods for treating an inflammatory disease include non-steroidal anti-inflammatory agents (NSAIDs); steroidal anti-inflammatory drugs, such as cortisol, dexamethasone, predinsone, prednisolone, methylprednisone, betamethasone, beclometasone, fludrocortisone acetate, deoxycorticosterone acetate, aldosterone, corticosterone and cortisone; agents useful for treating inflammatory bowel disease such as IL-10, steroids, and azulfidine; agents useful for treating rheumatoid arthritis such as methotrexate, azathioprine, cyclophosphamide, steroids and mycophenolate mofetil; agents for treating or preventing inflammatory bowel disease.

Other anti-inflammatory agents useful in the present methods for treating an inflammatory disease include, but are not limited to, rituximab, adalimumab, infliximab, etanercept, TACE inhibitors, muscarinic antagonists, kinase inhibitors, cytokine inhibitors and chemokine inhibitors.

Non-limiting examples of non-steroidal anti-inflammatory agents (NSAIDs) useful in the present methods for treating an inflammatory disease include salicylates such as aspirin, amoxipirin, benorilate, choline magnesium sulfate, diflunisal, faislamine, methyl salicylate, magnesium salicylate and salicyl saliciylate; arylalkanoic acids, such as diclofenac, aceclofenac, acemetacin, bromfenac, etodolac, indometacin, nabumetone, sulindac and tolmetin; profens, such as ibuprofen, carprofen, fenbufen, fenoprofen, flurbiprofen, ketoprofen, ketorolac, loxoprofen, naproxen, tiaprofenic acid and suprofen; fenamic acids, such as mefenamic acid and meclofenamic acid; pyrazolidine derivatives, such as phenylbutazone, azapropazone, metarnizole, oxyphenbutazone and sulfinprazone; oxicams, such as piroxicam, lornoxicam, meloxicam and tenoxicam; COX-2 inhibitors, such as celecoxib, rofecoxib, etoricoxib, lumiracoxib, parecoxib and valdecoxib; sulfonalides, such as nimesulfide; licofelone; omega-3 fatty acids; and PDE inhibitors.

In one embodiment, the NSAID is a profen or a salicylate.

In another embodiment, the NSAID is a COX-2 inhibitor.

In one embodiment, the at least one additional therapeutic agent comprises an anti-asthmatic agent.

Non-limiting examples of anti-asthmatic agents useful in the present methods for treating asthma include beta-2 adrenoceptor angoinsts, such as salmeterol, formoterol, bambuterol, albuterol, salbutamol, levalbuterol, terbutaline and bitolterol; ephedrine; ipatropium bromide; glucocorticoids, such as ciclesonide, beclomethasone, budesonide, funisolide, futicasone, mometasone and triamcinolone; leukotriene modifiers, such as montelukast, zafirlukast, pranlukast and zileuton; mast cell stabilizers, such as cromolyn and nedocromil; anticholinergics, such as ipatropium, glycopyrrolate, atropine, oxitropium and tiotropium; methylxanthines, such as theophylline and aminophylline; an antihistamine; an IgE, such as omalizumab; methotrexate; tianeptine; steroids such as prednisone, prednisolone, methylprednisone, dexamethasone and hydrocortisone; beta-agonists, such as epinephrine, isoetharine, isoproterenol and metaproterenol; inhalation anesthetics, such as isoflurane, halothane and enflurane; magnesium sulfate; heliox, which is a mixture of helium and oxygen; and expectorants, such as guaifenesin.

In one embodiment, the inflammatory disease treated using the combination therapies of the present invention is asthma. In another embodiment, the inflammatory disease is arthritis. In still another embodiment, the inflammatory disease is rheumatoid arthritis or osteoarthritis. In yet another embodiment, the inflammatory disease is COPD. In a further embodiment, the inflammatory disease is inflammatory bowel disease.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) are administered during a time when the additional therapeutic agent(s) exert their prophylactic or therapeutic effect, or vice versa.

In another embodiment, the one or more Compounds of Formula (I) and the least one additional therapeutic agent are administered in doses commonly employed when such agents are used as monotherapy for treating a Condition.

In another embodiment, the one or more Compounds of Formula (I) and the least one additional therapeutic agent are administered in doses lower than the doses commonly employed when such agents are used as monotherapy for treating a Condition.

In still another embodiment, the one or more Compounds of Formula (I) and the least one additional therapeutic agent act synergistically and are administered in doses lower than the doses commonly employed when such agents are used as monotherapy for treating a Condition.

In one embodiment, the one or more Compounds of Formula (I) and the least one additional therapeutic agent are present in the same composition. In one embodiment, this composition is suitable for oral administration. In another embodiment, this composition is suitable for intravenous administration.

The one or more Compounds of Formula (I) and the least one additional therapeutic agent can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the least one additional therapeutic agent may inhibit the resistance of a Condition to these agents.

In another embodiment, the additional therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Compound of Formula (I) and the other agent(s) for treating diseases or conditions listed above can be administered simultaneously or sequentially. This particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Compounds of Formula (I) and the least one additional therapeutic agent can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides methods for treating a Condition in a patient, comprising administering to the patient a composition comprising an effective amount of one or more Compounds of Formula (I) or a pharmaceutically acceptable salt, solvate, ester or prodrug thereof, and a pharmaceutically acceptable carrier. In another embodiment, the invention provides methods for treating a Condition in a patient, comprising administering to the patient more than one composition, each comprising an effective amount of one or more Compounds of Formula (I) or a pharmaceutically acceptable salt, solvate, ester or prodrug thereof, and a pharmaceutically acceptable carrier.

For preparing pharmaceutical compositions from the Compounds of Formula (I), inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Compound of Formula (I) is administered orally.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of one or more Compounds of Formula (I) and at least one additional therapeutic agents, the two active components may be co-administered simultaneously or sequentially, or a single pharmaceutical composition comprising one or more Compounds of Formula (I) and at least one additional therapeutic agents in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising an effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Compounds of Formula (I), or a pharmaceutically acceptable salt or solvate of the compound and an amount of at least one additional therapeutic agent listed above, wherein the combined amounts are effective for treating or preventing diabetes, a diabetic complication impaired glucose tolerance or impaired fasting glucosein a patient.

When the components of a combination therapy regime are to be administered in more than one composition, they can be provided in a kit comprising in a single package, one or more containers, each comprising one or more Compounds of Formula (I) in a pharmaceutically acceptable carrier, and a separate container comprising an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited herein, the entire disclosures of which are incorporated herein by reference.

What is claimed is:

1. A compound having the formula: 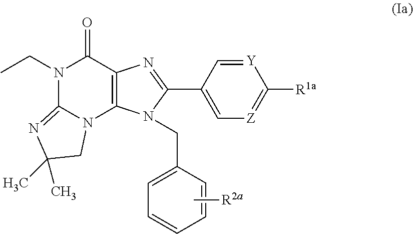
wherein:
Y is N or —C(R^{3a})—;
Z is N or —C(R^{3a})—;
R^{1a} is —O-alkyl, —O-haloalkyl, cyclohexyl, piperidinyl, pyridyl, pyridazinyl, pyrazinyl, phenyl, piperidinyl or oxadiazolyl, wherein a piperidinyl, pyridyl, pyridazinyl, pyrazinyl, phenyl, piperidinyl or oxadiazolyl group can be unsubstituted or optionally substituted with up to 2 groups, which can be the same or different, and are selected from alkyl, —O-alkyl, —CN, halo or —C(O)O-alkyl;
R^{2a} represents at least one —F group and up to a maximum of three —F groups; and
each occurrence of R^{1a} is independently H, F, OCHF₂, or —CN.

2. The compound of claim 1, wherein Y is N and Z is —C(R^{3a})—.

3. The compound of claim 2, wherein Z is N and Y is —C(R^{3a})—.

4. The compound of claim 1, wherein Y and Z are each N.

5. The compound of claim 2, wherein the ring containing Y and Z has the structure:

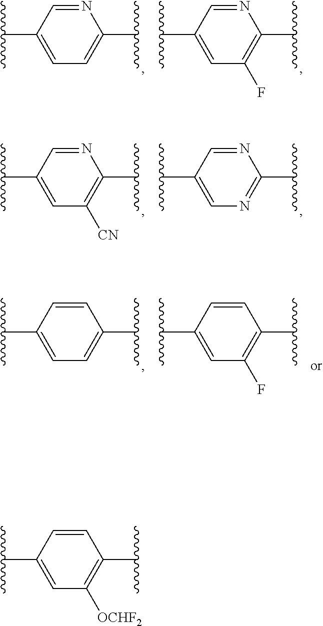

6. The compound of claim 1, wherein the ring substituted with R^{2a} has the structure:

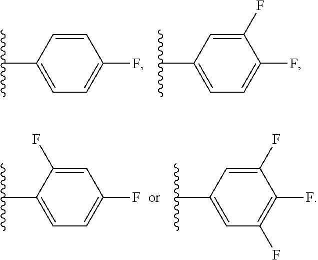

7. The compound of claim 1, wherein R^{1a} is methoxy or —OCHF₂.

8. The compound of claim 1, wherein R^{1a} is piperidinyl, which can be unsubstituted or optionally substituted with a —C(O)O-alkyl group.

9. The compound of claim 1, wherein RR^{1a} is pyridyl, which can be unsubstituted or optionally substituted with up to 2 groups, which can be the same or different, and are selected from alkyl, —O-alkyl and halo.

10. The compound of claim 1, wherein R^{1a} is pyridazinyl, which can be unsubstituted or substituted with an alkyl group.

11. The compound of claim 1, wherein R^{1a} is pyrazinyl, which can be unsubstituted or substituted with an alkyl group.

12. The compound of claim 1, wherein R^{1a} is phenyl, which can be unsubstituted or substituted with a —CN group.

13. The compound of claim 1, wherein R^{1a} is oxadiazolyl, which can be the same or different, and are selected from alkyl, —O-alkyl, —CN, halo and —C(O)O-alkyl.

14. A compound having the structure: 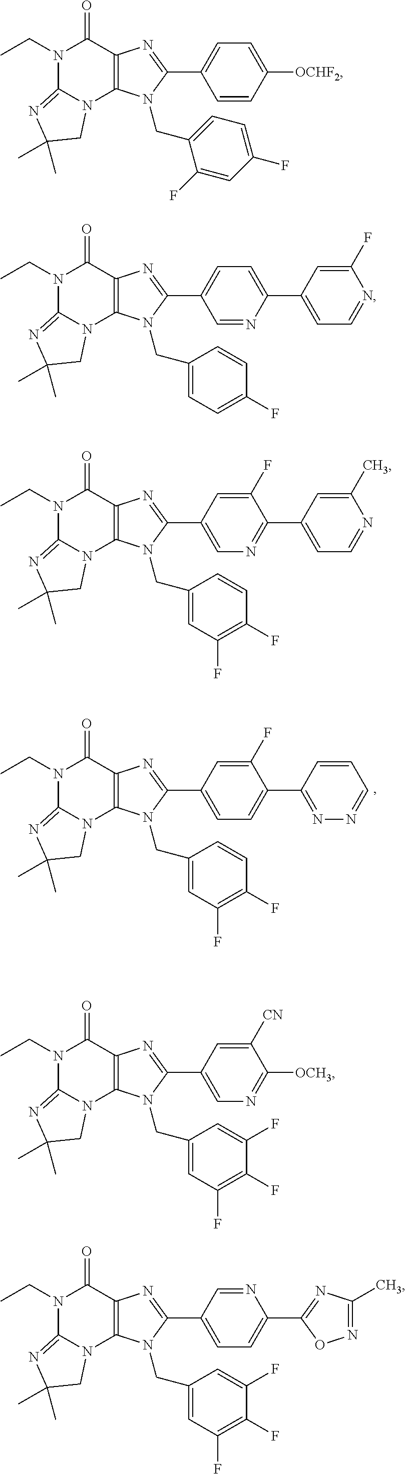 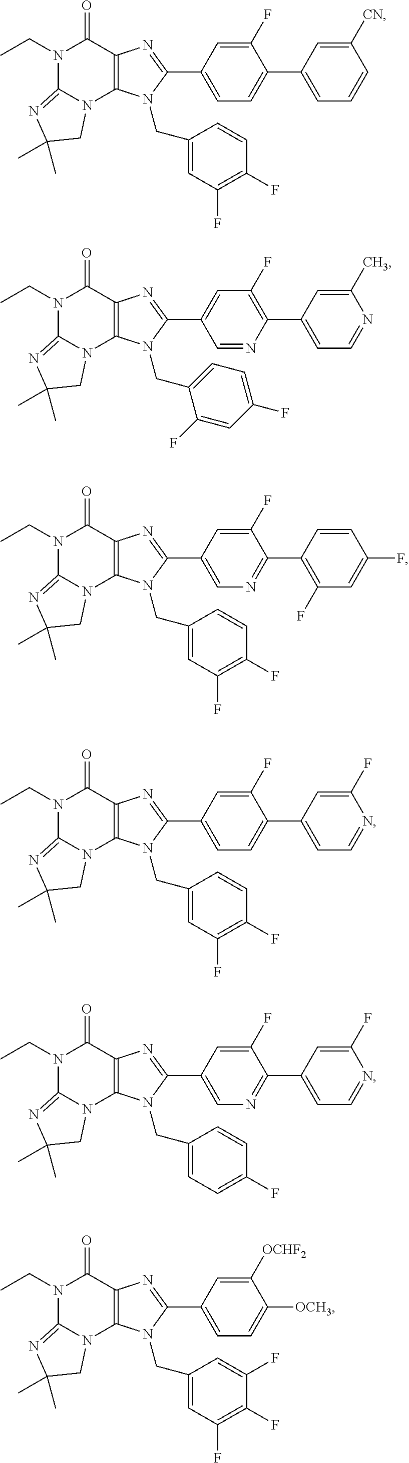 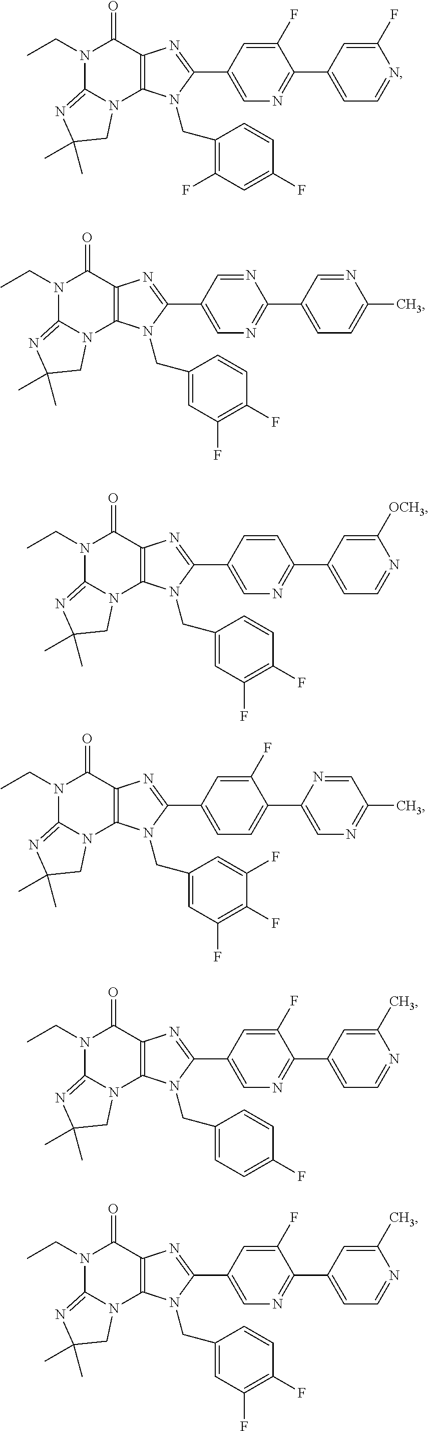 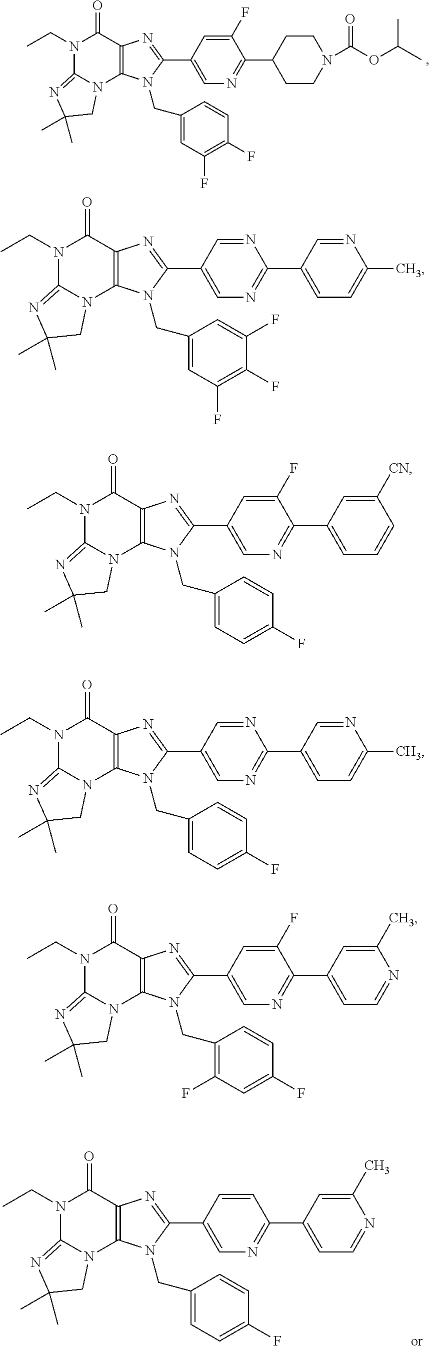 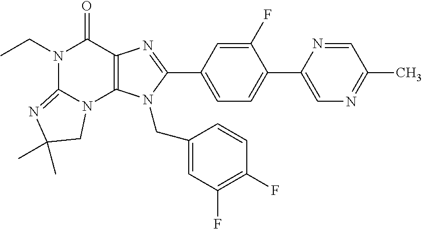
or a pharmaceutically acceptable salt, solvate, ester or prodrug thereof.

15. A composition comprising an effective amount of one or more compounds of claim 1, or a pharmaceutically acceptable salt, solvate, ester or prodrug thereof, and a pharmaceutically acceptable carrier.

16. The composition of claim 15, further comprising an effective amount of at least one additional therapeutic agent, wherein the additional therapeutic agent is an analgesic agent or an anti-inflammatory agent and wherein the additional agent is not a compound of claim 1.

17. A method for treating an inflammatory disease in a patient, the method comprising administering to the patient an effective amount of one or more compounds of claim 1 or a pharmaceutically acceptable salt, solvate, ester or prodrug thereof.

18. The method of claim 17, further comprising administering to the patient an additional anti-inflammatory agent, wherein the additional agent is not a compound of claim 1, and wherein the amounts administered are together effective to treat an inflammatory disease, wherein said inflammatory disease being treated is selected from the group consisting of rheumatoid arthritis, osteoarthritis, asthma, and chronic obstructive pulmonary disease.

19. A method for treating pain in a patient, the method comprising administering to the patient an effective amount of one or more compounds of claim 1 or a pharmaceutically acceptable salt, solvate, ester or prodrug thereof.

20. The method of claim 19, further comprising administering to the patient an additional analgesic agent, wherein the additional analgesic agent is not a compound of claim 1, and wherein the amounts administered are together effective to treat pain.
